(19) United States
(12) Patent Application Publication
(10) Pub. No.: US 2002/0142329 A1
(43) Pub. Date: Oct. 3, 2002

(54) Compositions and methods employing cleavable electrophoretic tag reagents

(75) Inventors: Tracy Matray, San Lorenzo, CA (US); Vincent Hernandez, Brookdale, CA (US); Sharat Singh, San Jose, CA (US)

Correspondence Address: PERKINS COIE LLP, P.O. BOX 2168, MENLO PARK, CA, 94026, US

(73) Assignee: Aclara BioSciences, Inc.

(21) Appl. No.: 10/008,573

(22) Filed: Nov. 9, 2001

Related U.S. Application Data
(63) Continuation-in-part of application No. 09/698,846, filed on Oct. 27, 2000.
Application No. 09/698,846 is a continuation-in-part of application No. 09/602,586, filed on Jun. 21, 2000.
Application No. 09/602,586 is a continuation-in-part of application No. 09/684,386, filed on Oct. 4, 2000.
Application No. 09/684,386 is a continuation-in-part of application No. 09/561,579, filed on Apr. 28, 2000, now abandoned.
Application No. 09/561,579 is a continuation-in-part of application No. 09/303,029, filed on Apr. 30, 1999, now Pat. No. 6,322,980.

(51) Int. Cl. C12Q 1/68; G01N 33/53; C07H 21/04; C07K 16/46

(52) U.S. Cl. 435/6; 435/7.1; 530/390.1; 536/24.3

(57) ABSTRACT

Probe sets for the multiplexed detection of the binding of, or interaction between, one or more ligands and target antiligands are provided. Detection involves the release of identifying tags as a consequence of target recognition. The probe sets include electrophoretic tag probes or e-tag probes, comprising a detection region and a mobility-defining region called the mobility modifier, both linked to a target-binding moiety. Target antiligands are contacted with a set of e-tag probes and the contacted antiligands are treated with a selected cleaving agent resulting in a mixture of e-tag reporters and uncleaved and/or partially cleaved e-tag probes. The mixture is exposed to a capture agent effective to bind to uncleaved or partially cleaved e-tag probes, followed by electrophoretic separation. In a multiplexed assay, different released e-tag reporters may be separated and detected providing for target identification. The methods employ compositions comprising luminescent molecules such as, for example, fluorescent molecules, which are modified to provide for electrophoretic properties that differ for each modified luminescent molecule while maintaining substantially the same absorption, emission and quantum yield properties of the original luminescent molecule. The compositions may be cleavably linked to binding molecules to form the e-tag probes.

### CROSS-REFERENCE TO RELATED APPLICATIONS

This application is a continuation-in-part (CIP) of 09/698,846, filed Oct. 27, 2000, which is a CIP of 09/602,586, filed Jun. 21, 2000, which, with 09/684,386, filed Oct. 04, 2000 are CIP's of 09/561,579, filed Apr. 28, 2000, which is a CIP of 09/303,029, filed Apr. 30, 1999, all of which are incorporated herein by reference in their entirety.

### FIELD OF THE INVENTION

The present invention relates to separable compositions, methods, and kits for use in multiplexed assay detection of the interaction between ligands and target antiligands. The invention finds particular application to the area of multiplexed assays for polypeptides and polynucleotides.

### BACKGROUND OF THE INVENTION

The need to determine many analytes or nucleic acid sequences (for example multiple pathogens or multiple genes or multiple genetic variants) in blood or other biological fluids has become increasingly apparent in many branches of medicine. Most multi-analyte assays, such as assays that detect multiple nucleic acid sequences, involve multiple steps, have poor sensitivity, a limited dynamic range (typically on the order of 2 to 100-fold differences and some require sophisticated instrumentation.

There is a need, therefore, for assay for multiple target molecules that has higher sensitivity, a large dynamic range (10³ to 10⁴-fold differences in target levels), a greater degree of multiplexing, and fewer and more stable reagents would increase the simplicity and reliability of multianalyte assays and reduce their costs.

### BRIEF DESCRIPTION OF THE RELATED ART

W. Clark Still, in U.S. Pat. No. 5,565,324 and in Accounts of Chem. Res., (1996) 29:155, uses a releasable mixture of halocarbons on beads to code for a specific compound on the bead that is produced during synthesis of a combinatorial library. Beads bearing a compound of interest are treated to release the coding molecules and the mixture is analyzed by gas chromatography with flame ionization detection.

U.S. Pat. No. 5,807,682 describes probe compositions for detecting a plurality of nucleic acid targets.

U.S. Pat. No. 6,008,379 discloses aromatic substituted xanthene dyes.

U.S. Pat. No. 6,080,852 discusses 4,7-dichlororhodamine dyes.

Unsymmetrical fluorescein derivatives are disclosed in U.S. Pat. No. 4,439,356.

Xanthene dyes having a fused (C) benzo ring are discussed in U.S. Pat. No. 4,945,171.

4,7-Dichlorofluorescein dyes as molecular probes are discussed in WO 94/05688.

U.S. Pat. No. 4,351,760 discloses alkyl substituted fluorescent compounds and polyamino acid conjugates.

U.S. Pat. No. 4,318,846 discusses ether substituted fluorescein polyamino acid compounds as fluorescers and quenchers. PCT WO 97/39064 discloses fluorinated xanthene derivatives.

U.S. Pat. No. 5,807,682 describes probe compositions for detecting a plurality of nucleic acid targets.

### SUMMARY OF THE INVENTION

In a broad aspect the present invention is directed to compositions comprising luminescent molecules such as, for example, fluorescent molecules, which either are, or are modified to provide for, electrophoretic properties that differ for each luminescent molecule while maintaining substantially the same absorption, emission and quantum yield properties of the original luminescent molecule. The compositions may be cleavably linked to binding molecules either by the modified portion or by other than the modified portion to form electrophoretic tag probes, which may be employed in methods for simultaneously determining multiple analytes in a sample suspected of containing the analytes.

One embodiment of the present invention is a set of electrophoretic tags wherein each member of the set comprises a mobility modifier, a detectable label and a target binding moiety. In accordance with the invention, at least two fluorescent compounds are independently employed as a detectable label in the set wherein the fluorescent compounds have substantially the same spectral properties and different mass and charge.

In another embodiment the invention concerns electrophoretic tag (e-tag) probes and sets of electrophoretic tag (e-tag) probes for detecting the binding of or interaction between each or any of a plurality of ligands and one or more target antiligands. The probes have the formula M—D—L—T and the set comprises j members, and each of the e-tag probes has the form: Mⱼ—D—L—Tⱼ, wherein (a) D is a detection group comprising a detectable label that is the same for all of the e-tag probes; (b) Tⱼ is a ligand capable of binding to or interacting with a target antiligand, (c) L is a bond or a linking group linking D and Tⱼ and comprising a cleavable linkage at the point of attachment to D or within L at a point that is common to all of the e-tag probes, wherein cleavage of the cleavable linkage produces an e-tag reporter of the form Mⱼ—D or Mⱼ—D—L′, where L′ is the residue of L attached to Mⱼ—D after such cleavage, and d) Mⱼ is a mobility modifier having a charge/mass ratio that imparts a unique and known electrophoretic mobility to a corresponding e-tag reporter, within a selected range of electrophoretic mobilities with respect to other e-tag reporters of the same form in the probe set.

In another embodiment the invention concerns fluorescers of the formula:

Fl—CH₂(CH₂)ₚ(CH₂(O CH₂(CH₂)_{q}CH₂)ₜOH

Fl is a fluorescer such as, for example, a fluorescein, a rhodamine, and the like and so forth, p is 1 to about 50, q is 1 to about 4 and t is 0 to about 5. Fluoresceins include substituted fluoresceins and other xanthenes and rhodamines include substituted rhodamines and other similar compounds.

In the methods of the invention a combination is provided comprising the sample and an electrophoretic tag probe set as described above. The electrophoretic tag probe, or e-tag probe, is involved in a binding event that is related to the presence of the analyte in the sample. The combination is treated with reagents under conditions sufficient to release the releasable portion, forming an e-tag reporter. The presence and/or amount of the released e-tag reporter is detected and is related to the presence and/or amount of the analyte in the sample.

### BRIEF DESCRIPTION OF THE DRAWINGS

FIG. 1 depicts fluorescent compounds of the present invention wherein EX is excitation wavelength, EM is wavelength of emission and RQY is quantum yield and the charge for each of the compounds is −3.

FIG. 2 depicts a general formula for resorcinols that may be used in preparing the compounds of the present invention.

FIG. 3 depicts a general formula for phthalic acid anhydrides that may be used in preparing the compounds of the present invention.

FIG. 4 depicts a general formula for phthalic acids that may be used in preparing the compounds of the present invention.

FIG. 5 depicts one approach to the synthesis of 2-methyl-4-chlororesorcinol.

FIG. 6 depicts one approach to the synthesis of AMD-S 001.

FIG. 7 depicts one approach to the synthesis of dichlorotrimellitic acid.

FIG. 8 depicts one approach to the synthesis of AMD-S 002.

FIG. 9 depicts examples of resorcinols that may be used in the synthesis of fluorescent compounds of the invention.

FIG. 10 depicts FAM wherein EX is excitation wavelength, EM is wavelength of emission and RQY is quantum yield and the charge for the compounds is −3.

FIG. 11 depicts an electropherogram of 6-FAM.

FIG. 12 depicts an electropherogram of 6-FAM and AMD-S 002.

FIG. 13 depicts an electropherogram of 6-FAM and AMD-S 001.

FIG. 14 depicts compounds in accordance with one aspect of the present invention.

FIG. 15 defines abbreviations used in depicting the compounds of FIG. 14.

### DESCRIPTION OF THE SPECIFIC EMBODIMENTS

I. Definitions

The discussion in this application may be viewed in reference to U.S. pat. applications Ser. Nos. 09/698,846, filed Oct. 27, 2000, which is a CIP of 09/602,586, filed Jun. 21, 2000, which, with 09/684,386, filed Oct. 04, 2000 are CIP's of 09/561,579, filed Apr. 28, 2000, which is a CIP of 09/303,029, filed Apr. 30, 1999, all of which were incorporated herein by reference in their entirety.

In defining the terms below, it is useful to consider the makeup of the “electrophoretic probes” that form part of the invention and/or are used in practicing the method of the invention. An electrophoretic probe has four basic components or moieties: (i) a detection group or moiety, (ii) a mobility modifier, (iii) a target-binding moiety, and (iv) a linking group that links the mobility modifier and detection group to the target-bonding moiety. These terms will first be examined in the context of the functioning of the electrophoretic probes in the invention, then more fully defined by their structural features.

The function of an electrophoretic probe in the invention is first to interact with a target, such as a single-stranded nucleic acid, a protein, a ligand-binding agent, such as an antibody or receptor, or an enzyme, e.g., as an enzyme substrate. The “portion”, “region” or “moiety” of the probe which binds to the target is the “target-binding moiety” or “target-binding region” or “target-binding portion” (“T”). After the target-binding moiety of an electrophoretic probe binds to a target, and typically as a result of such binding, the linking group of the electrophoretic probe may be cleaved to release an “electrophoretic tag” or “e-tag” or “e-tag reporter” that has a unique mass or charge-to-mass ratio, rendering such e-tags separable by, for example, electroseparation or mass spectrometry. In one embodiment the e-tags have a unique electrophoretic mobility in a defined electrophoretic system. The e-tag reporter is composed of the detection group, mobility modifier, and any residue of the linking group that remains associated with released reporter e-tag after cleavage. Therefore, the second function of the electrophoretic probe is to release an e-tag reporter, which can be identified according to its unique and known electrophoretic mobility.

According to an important feature of the invention, there is provided a set of electrophoretic probes, each of which has a unique target-binding moiety and an associated “e-tag moiety” that imparts to the associated e-tag reporter, a unique electrophoretic mobility by virtue of a unique charge to mass ratio. In general, the unique charge to mass ratio of an e-tag moiety is due to the chemical structure of the mobility modifier, since the detection group and linking-group residue (if any) are generally common to any set of electrophoretic probes. However, it is recognized that the detection group can make unique charge and/or mass contributions to the e-tag reporters as well. For example, a set of electrophoretic probes may be made up of a first subset having a group of mobility modifiers that impart unique electrophoretic mobilities to the subset in combination with a detection group having one defined charge and/or mass, and a second subset having the same group of mobility modifiers in combination with a second detection group with a different charge and/or mass, thus to impart electrophoretic mobilities which are unique among both subsets.

The different target-binding moieties in a set of electrophoretic probes are typically designated “Tⱼ”, where the set of probes contains n members, and each Tⱼ, j=1 to j=n is different, i.e., will bind specifically and/or with unique affinities to different targets. A set of electrophoretic probes of the invention typically includes at least about 5 members, i.e., n is preferably 5 or more, typically 10-100 or more.

A “reporter moiety” “R” or a “detection group” “D” are equivalent terms referring to a chemical group or moiety that is capable of being detected by a suitable detection system, particular in the context of detecting molecules containing the detection group after or during electrophoretic separation. The detection group in accordance with the present invention is a fluorescent compound as disclosed herein. The fluorescent compounds can be readily detected during or after electrophoretic separation of molecules by illuminating the molecules with a light source in the excitation wavelength and detecting fluorescence emission from the irradiated molecules. Exemplary fluorescent compounds will be given below. As noted above, the detection group is typically common among a set or subset of different electrophoretic probes, but may also differ among probe subsets, contributing to the unique electrophoretic mobilities of the released e-tag reporter.

The “mobility modifier” “M” is a generally a chemical group or moiety that is designed to have a particular charge to mass ratio, and thus a particular electrophoretic mobility in a defined electrophoretic system. Exemplary types of mobility modifiers are discussed below. In a set of n electrophoretic probes, each unique mobility modifier is designated Mⱼ, where j=1 to n, as above. The mobility modifier may be considered to include a mass-modifying region and/or a charge-modifying region or a single region that acts as both a mass- and charge-modifying region. The mobility modifying region may also be referred to as M*, C*, L, a bond, a linking group, a mobility/mass identifying region or “mir”, a charge-imparting moiety and a mobility region.

The detection group and mobility modifier in the electrophoretic probe form an “e-tag moiety,” which is linked to the target-binding moiety by a “linking group.” The linking group may be only a covalent bond that is cleavable under selected cleaving conditions, or a chemical moiety or chain, such as, for example, a nucleotide and associated phosphodiester bond, an oligonucleotide with an internal cleavable bond, an oligopeptide, or an enzyme substrate, that contains a cleavable chemical bond. Cleavage typically occurs as the result of binding of the probe to the target, which is followed by enzyme or catalyzed cleavage of the linking-group bond or other type of cleavage depending on the nature of the cleavable linkage. The linking group is referred to herein as “L.”

The linking group may or may not contribute a linking-group “residue” to the released e-tag reporter, also dependent on the nature of the linking group and the site of cleavage. For example, where the linking group is a covalent bond, or cleavage of the linking group occurs immediately adjacent the “e-tag moiety”, the linking group leaves no residue, i.e., will not contribute additional mass and charge to the released e-tag reporter. Similarly, where the linking group is a chemical group or chain that is cleaved internally or immediately adjacent the target-binding moiety, cleavage of the linking group leaves a residual mass and possible charge, contribution to the released e-tag reporter. In general, this contribution will be relatively small, and the same for each different released e-tag (assuming a common linking group within the probe set). As such, generally, the residue will not effect the relative electrophoretic mobilities of the released e-tag reporters, nor the ability to resolve the e-tag reporters into electrophoretic species that can be uniquely identified.

The following definitions are to be understood in the context of the above function of the various components of electrophoretic probes and e-tag reporters. In some case, structure designations based on different lettering schemes are employed, and the equivalency between or among structures with different lettering schemes will be understood by those skilled in the art, in view of the intended function of the structure being referred to.

An “electrophoretic probe” refers to one of a set of probes of the type described above having unique target-binding moieties and associated e-tag moieties. The probes are variously expressed by the following equivalent forms herein:

(a) (D, Mⱼ)—L—Tⱼ, or (D, Mⱼ)—N—Tⱼ, where D is a detection moiety, Mⱼ is the jth mobility modifier, Tj is the jth target binding agent, and the linking group is represented by L and by N (when the linking group is the 5′-terminal nucleotide of an oligonucleotide target-binding moiety). In this and the following structural designations, (D, Mⱼ)— indicates that either the detection group or the mobility modifier is joined to the linking group, i.e., either (D, Mⱼ) or (Mⱼ, D)—.

(b) (R, Mⱼ)—L—Tⱼ, or (R, Mⱼ)—N—Tⱼ, where R is a detection moiety or reporter group, and Mⱼ, Tⱼ, and L and N are as in (a).

(c) R—L—T or L—R—T, where R is a label, particularly a fluorescer, L is a mir, a bond or a linking group, where L and the regions to which L is attached provide for the variation in mobility of the e-tags. T comprises a portion of the target-binding region, particularly a nucleoside base, purine or pyrimidine, and is the base, a nucleoside, nucleotide or nucleotide triphosphate, an amino acid, either naturally occurring or synthetic, or other functionality that may serve to participate in the synthesis of an oligomer, when T is retained, and is otherwise a functionality resulting from the cleavage between L, the mir, and the target-binding region. (in the corresponding e-tag reporter).

A “set” or “group”, “plurality” or “library” of electrophoretic probes refers to a plurality of electrophoretic probes having typically at least five, typically 10-100 or more probes with different unique target-binding moieties and associated e-tag moieties.

As used herein, the term “electrophoretic tag probe set” or “e-tag probe set” refers to a set of probes for use in detecting each or any of a plurality of known, selected target nucleotide sequences, or for detecting the binding of, or interaction between, each or any of a plurality of ligands and one or more target antiligands.

The term “target-binding moiety” or “Tⱼ” refers to the component of an e-tag probe that participates in recognition and specific binding to a designated target. The target-binding moiety may also be referred to as T or T′, or may be defined based on the type of target, e.g., as a snp detection sequence or an oligonucleotide detection sequence.

In one application of this embodiment, the e-tag probe is referred to as a snp detection sequence, a fluorescence snp detection sequence or an oligonucleotide detection sequence.

In another generalized embodiment for use in detection of non-nucleic acid targets, the target-binding moiety, Tⱼ, is or includes a ligand capable of binding to or interacting with a target antiligand and L is a linking group connected to Tⱼ by a bond that is cleavable by a selected cleaving agent when the probe is bound to or interacting with the target antiligand. L may also be referred to as “L”, a terminal linking region, a terminal linking group.

“Electrophoretic tag” refers to a composition or reagent for unique identification of an entity of interest during separation. An e-tag has the fundamental structure given as (D, Mⱼ)—L, where D and Mⱼ are the detection group and jth mobility modifier, as defined above, and L is the linking group, and in particular, the bond or residue of the linking group remaining after cleavage. Here the e-tag moiety (D, Mⱼ) is intended to include both of the structures D—Mⱼ—L and Mⱼ—D—L. Other equivalent forms of expressing the e-tag are: (R, Mⱼ), (R, M), R—L or L—R where R is a reporter group, Mⱼ or M is a mobility modifier and L is a mobility identifying region (mir), a bond or a linking group.

For purposes of clarity, the concept of an electrophoretic tag is consistently referred to herein as an “e-tag”, however various references to “Etag”, “ETAG”, “eTAG” and “eTag” may be made when referring to an electrophoretic tag. As used herein, the term “electrophoretic tag probe” or “e-tag probe” refers to a reagent used for target recognition, which comprises an e-tag and a target-binding moiety. Upon interaction with the corresponding target, the e-tag undergoes a change resulting in the release of an e-tag reporter. Such an e-tag probe may also be referred to as a binding member.

E-tag probes of the invention find utility in performing multiplexed for detection/analysis of targets including, but not limited to nucleic acid detection, such as sequence recognition, snp detection, transcription analysis or mRNA determination, allelic determination, mutation determination, HLA typing or MHC determination and haplotype determination, in addition to detection of other ligands, such as proteins, polysaccharides, etc.

As used herein, the term “e-tag reporter” refers to the cleavage product generated as a result of the interaction between an e-tag probe and its target. In one representation, an e-tag reporter comprises the e-tag plus a residual portion of the target binding moiety (Tⱼ) (where, as in the nucleotide example, above, one or more nucleotides in the target-binding moiety contain the cleavable linking group), or a residual portion of the linking group (when the latter is considered separate from the target-binding moiety). In another embodiment, the e-tag does not retain any of the target-binding moiety. E-tag reporters can be differentiated by electrophoretic mobility or mass and are amenable to electrophoretic separation and detection, although other methods of differentiating the tags may also find use.

An e-tag reporter resulting from the interaction of an e-tag probe and a nucleic acid target typically has the form (D, Mⱼ)—N, where N is as defined above, the 5′-end terminal nucleotide of a target-binding oligonucleotide.

An e-tag reporter resulting from the interaction of an e-tag probe used to detect the binding of or interaction between a ligand and an antiligand typically has the form (D, Mⱼ)—L′. D and Mⱼ are defined above and L′ is the residue of L that remains attached to (D, Mⱼ) after an e-tag reporter is cleaved from the corresponding e-tag probe.

E-tag reporters may also be described as electrophoretic tags or eTags for use in electrophoresis, released eTags, released e-tags, etc. The e-tag for use in electrophoresis may also be represented by the formula: R—L—T, as described above, where T is retained, and is otherwise a functionality resulting from the cleavage between L, the mir, and the target-binding region.

As used herein, the term “binding event” generally refers to the binding of the target-binding moiety of an e-tag probe to its target. By way of example, such binding may involve the interaction between complementary nucleotide sequences or the binding between a ligand and target antiligand.

As used herein, the term “capture ligand”, refers to a group that is typically included within the target binding moiety or portion of an e-tag probe and is capable of binding specifically to a “capture agent” or receptor. The interaction between such a capture ligand and the corresponding capture agent may be used to separate uncleaved e-tag probes from released e-tag reporters.

II. Compositions of the Invention

The subject invention provides compositions and methods for improved analysis of complex mixtures and may be employed, for example, in the simultaneous identification of a plurality of entities, such as nucleic acid or amino acid sequences, snp's, alleles, mutations, proteins, haptens, protein family members, expression products, etc., analysis of the response of a plurality of entities to an agent that can affect the mobility of the entities, and the like. Libraries of differentiable compounds are provided, where the compounds comprise a mobility-identifying region (including mass-identifying region) (“mir” or “mobility modifier”), that provides for ready identification by electrophoresis or mass spectrometry (differentiation by mobility in an electrical field or magnetic field), by itself or in conjunction with a detectable label. Depending on the determination the product may also include one or more nucleotides or their equivalent, one or more amino acids or their equivalent, a functionality resulting from the release of the target-binding region or moiety or a modified functionality as a result of the action of an agent on the target-binding moiety. The mobility-identifying region or mobility modifier may be designated as a mobility modifier given that it provides for ready identification by electrophoresis, by itself or in conjunction with a detectable label.

The methodology involves employing detectable tags that can be differentiated by electrophoretic mobility or mass. The tags comprise mobility-identifying regions joined to a moiety that will undergo a change to produce a product. Depending on the nature of the change, the change may involve a change in mass and/or charge of the mobility modifier, the release of the mobility modifier from all or a portion of the target-binding moiety or may provide for the ability to sequester the mobility modifier from the starting material for preferential release of the mobility modifier. The differentiable tags, whether identified by electrophoresis or mass spectrometry, comprising the mobility modifier, with or without the detectable label and a portion of the target-binding moiety will be referred to as “e-tags.”

Such differentiable e-tags, comprising the e-tag with or without a portion of the target-binding moiety for use in detection may be conveniently referred to as “e-tag reporters”. The e-tag reporters are generated as the result of the interaction between an e-tag probe (which comprises an e-tag joined to a target-binding moiety) and a corresponding target.

In addition, the subject invention employs a variety of reagent systems, where a binding event results in a change in mobility of the e-tag. The binding event is between a target-binding moiety and a target, and the reagent system recognizes this event and changes the nature of the e-tag containing target-binding moiety, so that the mobility and/or mass of the product is different from the starting material. The reagent system will frequently involve an enzyme and the reagent system may comprise the target. The effect of the reagent system is to make or break a bond by physical, chemical or enzymatic means. Each of the products of the different e-tag containing target-binding moieties can be accurately detected, so as to determine the occurrence of the binding event. Following the binding event, one or more reaction products are produced that exhibit mobilities different from the e-tag probe or probes from which the reaction products derive. The released form of the e-tag or the e-tag reporter exhibits a different mobility and/or mass than the e-tag from which it derives.

The subject invention may be used for a variety of multiplexed analyses involving the action of one or more agents on a plurality of reagents comprising the mobility modifier and a target-binding moiety that undergoes a change as a result of a chemical reaction, resulting in a change in mobility of the product as compared to the starting material. The reaction may be the result of addition or deletion in relation to the target-binding moiety, so that the resulting product may be sequestered from the starting material. The subject systems find use in nucleic acid and protein analyses, reactions, particularly enzyme reactions, where one or more enzymes are acting on a group of different potential or actual substrates, and the like.

A system is provided for the simultaneous multiplexed determination of a plurality of events employing electrophoresis to distinguish the events, comprising an electrophoretic device for electrophoretic separation and detection, a container containing a first set of first agents, referred to as “e-tags,” comprising differing mobility regions and a second reagent composition comprising at least one active second agent, under conditions where said second agent modifies at least one member of said first agent set resulting in a change of electrophoretic mobility of said at least one member to provide a modified member retaining said mobility region, and transfer of said at least one modified member to said electrophoretic device for separation and detection of said at least one modified member. The electrophoretic device may be connected to a data processor for receiving and processing data from the device, as well as operating the electrophoretic device.

The first set of first agents are considered to be “e-tag probes,” and the modified members that retain the mobility region or mobility modifying region and are subjected to analysis are referred to as “e-tag reporters”. In general, the e-tag probes comprise a mobility modifying region that is joined to a target binding moiety by a linker to a detection group to which the mobility modifying region is attached. The linker may include or be a reactive functionality, a cleavable linkage, a bond that may or may not be releasable or a group for joining to one or more of the other regions.

The systems are based on having libraries available comprising a plurality of e-tags that comprise at least a plurality of different mobility-identifying regions, so as to be separable by electrophoresis with the entities to which the mobility-identifying regions are attached. The mobility-identifying regions are retained in the product of the reaction, where the product is modified by the gain and/or loss of a group that changes the mass and may also change the charge of the product, as compared to the starting material. In some instances, the mobility-identifying region may be joined to a target-binding moiety by a cleavable bond, so that the mobility-identifying region is released for analysis subsequent to the modification of the target-binding moiety, e.g. complex formation.

In one aspect, the subject assays are predicated on having a reagent that has a high affinity for a reciprocal binding member, the analyte. Usually, the binding affinity will be at least about 10⁻⁷M⁻¹, more usually, at least about 10⁻⁸M⁻¹. For the most part, the reagents will be receptors, which includes antibodies, IgA, IgD, IgG, IgE and IgM and subtypes thereof, enzymes, lectins, nucleic acids, nucleic acid binding proteins, or any other molecule that provides the desired specificity for the analyte in the assay. The antibodies may be polyclonal or monoclonal or mixtures of monoclonal antibodies depending on the nature of the target composition and the targets. The targets or analytes may be any molecule, such as small organic molecules of from about 100 to 2500 Da, poly(amino acids) including peptides of from about 3 to 100 amino acids and proteins of from about 100 to 50,000 or more amino acids, saccharides, lipids, nucleic acids, etc., where the analytes may be part of a larger assemblage, such as a cell, microsome, organelle, virus, protein complex, chromosome or fragment thereof, nucleosome, etc.

A. Electrophoretic Tags

An e-tag will be a molecule, which is labeled with a directly detectable label or can be made so by functionalization. The electrophoretic tags will be differentiated by their electrophoretic mobility, usually their mass/charge ratio, to provide different mobilities for each electrophoretic tag. Although in some instances the electrophoretic tags may have identical mass/charge ratios, such as oligonucleotides but differ in size or shape and therefore exhibit different electrophoretic mobilities under appropriate conditions. Therefore, the e-tags will be amenable to electrophoretic separation and detection, although other methods of differentiating the tags may also find use. The mobility modifier of the e-tag is joined to the detectable label by a non-cleavable linkage to provide the e-tag or e-tag reporter. The detectable label of the e-tag reporter may be joined to any convenient site on the target binding moiety to form the e-tag probe without interfering with the synthesis, release and binding of the e-tag labeled reagent. For nucleotides, the e-tag label may be bound to a site on the base, either an annular carbon atom or a hydroxyl or amino substituent. For proteins, the e-tag label may be bound to multiple sites either on the protein or through the intermediacy of a hub nucleus.

In mass spectrometry, the e-tags may be different from the e-tags used in electrophoresis, since the e-tags do not require a label, or a charge. Thus, these e-tags may be differentiated solely by mass, which can be a result of atoms of different elements, isotopes of such elements, and numbers of such atoms.

Electrophoretic tags are small molecules (molecular weight of 150 to 10,000), usually other than oligonucleotides, which can be used in any measurement technique that permits identification by mass, e.g. mass spectrometry, and or mass/charge ratio, as in mobility in electrophoresis. Simple variations in mass and/or mobility of the electrophoretic tag leads to generation of a library of electrophoretic tags, that can then be used to detect multiple target molecules such as snp's or multiple target sequences or multiple proteins. The electrophoretic tags are easily and rapidly separated in free solution without the need for a polymeric separation media. Quantitation is achieved using internal controls. Enhanced separation of the electrophoretic tags in electrophoresis is achieved by modifying the tags with positively charged moieties.

The e-tags are a group of reagents that have a mobility modifier and that provide for unique identification of an entity of interest. The mobility modifier of the e-tags can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility modifier will have from 0 to 40, more usually from 0 to 30 heteroatoms, which in addition to the heteroatoms indicated above will include halogen or other heteroatom. The total number of atoms other than hydrogen will generally be fewer than 200 atoms, usually fewer than 100 atoms. Where acid groups are present, depending upon the pH of the medium in which the mobility modifier is present, various cations may be associated with the acid group. The acids may be organic or inorganic, including carboxyl, thionocarboxyl, thiocarboxyl, hydroxamic, phosphate, phosphite, phosphonate, sulfonate, sulfinate, boronic, nitric, nitrous, etc. For positive charges, substituents will include amino (includes ammonium), phosphonium, sulfonium, oxonium, etc., where substituents will generally be aliphatic of from about 1-6 carbon atoms, the total number of carbon atoms per heteroatom, usually be less than about 12, usually less than about 9. The mobility modifier may be neutral or charged depending on the other regions to which the mobility modifier is attached, at least one of the regions having at least one charge. Neutral mobility modifiers will generally be polymethylene, halo- or polyhaloalkylene or aralkylene (a combination of aromatic—includes heterocyclic—and aliphatic groups), where halogen will generally be fluorine, chlorine, bromine or iodine, polyethers, particularly, polyoxyalkylene, wherein alkyl is of from 2-3 carbon atoms, polyesters, e.g. polyglycolide and polylactide, dendrimers, comprising ethers or thioethers, oligomers of addition and condensation monomers, e.g. acrylates, diacids and diols, etc. The side chains include amines, ammonium salts, hydroxyl groups, including phenolic groups, carboxyl groups, esters, amides, phosphates, heterocycles, particularly nitrogen heterocycles, such as the nucleoside bases and the amino acid side chains, such as imidazole and quinoline, thioethers, thiols, or other groups of interest to change the mobility of the e-tag. The mobility modifier may be a homooligomer or a heterooligomer, having different monomers of the same or different chemical characteristics, e.g., nucleotides and amino acids. Desirably neutral mass differentiating groups will be combined with short charged sequences to provide the mobility modifier.

The charged mobility modifiers will generally have only negative or positive charges, although, one may have a combination of charges, particularly where a region to which the mobility modifier is attached is charged and the mobility modifier has the opposite charge. The mobility modifiers may have a single monomer that provides the different functionalities for oligomerization and carry a charge or two monomers may be employed, generally two monomers. One may use substituted diols, where the substituents are charged and dibasic acids. Illustrative of such oligomers is the combination of diols or diamino, such as 2,3-dihydroxypropionic acid, 2,3-dihydroxysuccinic acid, 2,3-diaminosuccinic acid, 2,4-dihydroxyglutaric acid, etc. The diols or diamino compounds can be linked by dibasic acids, which dibasic acids include the inorganic dibasic acids indicated above, as well as dibasic acids, such as oxalic acid, malonic acid, succinic acid, maleic acid, fumaric acid, carbonic acid, etc. Instead of using esters, one may use amides, where amino acids or diamines and diacids may be employed. Alternatively, one may link the hydroxyls or amines with alkylene or arylene groups.

By employing monomers that have substituents that provide for charges or which may be modified to provide charges, mobility modifiers may be obtained having the desired mass/charge ratio. For example, by using serine or threonine, the hydroxyl groups may be modified with phosphate to provide negatively charged mobility modifiers. With arginine, lysine and histidine, positively charged mobility modifiers may be obtained. Oligomerization may be performed in conventional ways to provide the appropriately sized mobility modifier. The different mobility modifiers having different orders of oligomers, generally having from 1 to 20 monomeric units, more usually about 1 to 12, where a unit intends a repetitive unit that may have from 1 to 2 different monomers. For the most part, oligomers are used with other than nucleic acid target-binding moieties. The polyfunctionality of the monomeric units provides for functionalities at the termini that may be used for conjugation to other moieties, so that an available functionality for reaction may be used to provide a different functionality. For example, a carboxyl group may be reacted with an aminoethylthiol, to replace the carboxyl group with a thiol functionality for reaction with an activated olefin.

By using monomers that have 1-3 charges, one may employ a low number of monomers and provide for mobility variation with changes in molecular weight. Of particular interest are polyolpolycarboxylic acids having from about two to four of each functionality, such as tartaric acid, 2,3-dihydroxyterephthalic acid, 3,4-dihydroxyphthalic acid, Δ⁵-tetrahydro-3,4-dihydroxyphthalic acid, etc. To provide for an additional negative charge, these monomers may be oligomerized with a dibasic acid, such as a phosphoric acid derivative to form the phosphate diester. Alternatively, the carboxylic acids could be used with a diamine to form a polyamide, while the hydroxyl groups could be used to form esters, such as phosphate esters, or ethers such as the ether of glycolic acid, etc. To vary the mobility, various aliphatic groups of differing molecular weight may be employed, such as polymethylenes, polyoxyalkylenes, polyhaloaliphatic or -aromatic groups, polyols, e.g. sugars, where the mobility will differ by at least about 0.01, more usually at least about 0.02 and more usually at least about 0.5. Alternatively, the libraries may include oligopeptides for providing the charge, particularly oligopeptides of from 2-6, usually 2-4 monomers, either positive charges resulting from lysine, arginine and histidine or negative charges, resulting from aspartic and glutamic acid. Of course, naturally occurring amino acids need not be used, unnatural or synthetic amino acids, such as taurine, phosphate substituted serine or threonine, S-α-succinylcysteine, co-oligomers of diamines and amino acids, etc., may be employed.

Where the e-tags are used for mass detection, as with mass spectrometry, the e-tags need not be charged but merely differ in mass, since a charge will be imparted to the e-tag reporter by the mass spectrometer. Thus, one could use the same or similar monomers, where the functionalities would be neutral or made neutral, such as esters and amides of carboxylic acids. Also, one may vary the e-tags by isotopic substitution, such as ²H, ¹⁸O, ¹⁴C, etc.

While the charge to mass ratio is an important aspect in differences between e-tag reporters and in particular between mobility modifiers, it is not the only manner by which e-tag reporters may differ from one another. The e-tag reporters may differ by overall topography of the e-tag reporter, i.e., the detection group and the mobility modifier. For example, the mobility modifier may comprise a rigidifier, or substituent that comprises one or more rings. Examples include an aryl moiety, such as, e.g., phenyl, benzyl, naphthyl, and so forth, a cycloalkyl moiety where alkyl is 3 to 20 carbon atoms such as, e.g., cyclopentyl, cyclohexyl and so forth, and the like. Any rigidifier may be employed that imparts a coefficient of drag for the e-tag reporter and, thus, results in a species with separation characteristics that differ from the separation characteristics of other e-tag reporters. Substituted aryl groups can serve as both mass- and charge-modifying regions. Various functionalities may be substituted onto a ring such as an aromatic group, e.g. phenyl, to provide mass as well as charges to the e-tag reporter in addition to rigidification.

The e-tag reporter may be linked to the target binding moiety through the detection group by a bond that may be cleavable thermally, photolytically or chemically. In some situations there may be an interest in cleaving the e-tag from the target-binding moiety in situations where cleavage of the target-binding moiety results in significant cleavage at other than the desired site of cleavage, resulting in satellite cleavage products, such as di- and higher oligonucleotides and this family of products interferes with the separation and detection of the e-tags. However, rather than requiring an additional step in the identification of the tags by releasing them from the base to which they are attached, one can modify the target binding sequence to minimize obtaining cleavage at other than the desired bond, for example, the ultimate or penultimate phosphate link in a nucleic acid sequence. For immunoassays involving specific binding members, bonding of the e-tag will usually be through a cleavable bond to a convenient functionality, such as carboxy, hydroxy, amino or thiol, particularly as associated with proteins, lipids and saccharides.

The nature of the releasable or cleavable link between the e-tag reporter and the target binding moiety may be varied widely. Numerous linkages are available, which are thermally, photolytically or chemically labile. See, for example, U.S. Pat. No. 5,721,099. Where detachment of the product from all or a portion of the target-binding moiety is desired, there are numerous functionalities and reactants, which may be used. Conveniently, ethers may be used, where substituted benzyl ether or derivatives thereof, e.g. benzhydryl ether, indanyl ether, etc. may be cleaved by acidic or mild reductive conditions. Alternatively, one may employ beta-elimination, where a mild base may serve to release the product. Acetals, including the thio analogs thereof, may be employed, where mild acid, particularly in the presence of a capturing carbonyl compound, may serve. By combining formaldehyde, HCl and an alcohol moiety, an α-chloroether is formed. This may then be coupled with an hydroxy functionality to form the acetal. Various photolabile linkages may be employed, such as o-nitrobenzyl, 7-nitroindanyl, 2-nitrobenzhydryl ethers or esters, etc.

For a list of cleavable linkages, see, for example, Greene and Wuts, Protective Groups in Organic Synthesis, 2^{nd} ed. Wiley, 1991. The versatility of the various systems that have been developed allows for broad variation in the conditions for attachment of the e-tag entities.

Various functionalities for cleavage are illustrated by: silyl groups being cleaved with fluoride, oxidation, acid, bromine or chlorine; o-nitrobenzyl with light; catechols with cerium salts; olefins with ozone, permanganate or osmium tetroxide; sulfides with singlet oxygen or enzyme catalyzed oxidative cleavage with hydrogen peroxide, where the resulting sulfone can undergo elimination; furans with oxygen or bromine in methanol; tertiary alcohols with acid; ketals and acetals with acid; α- and β-substituted ethers and esters with base, where the substituent is an electron withdrawing group, e.g., sulfone, sulfoxide, ketone, etc., and the like.

In one aspect of the present invention the electrophoretic tags in a set of tags utilize detectable labels that differ in mass and charge but have substantially the same spectral properties of excitation wavelength and emission wavelength. By the phrase “substantially the same spectral properties” is meant that the excitation wavelength and the emission wavelength of two compounds are within about 5% of one another, usually, within about 3% of one another, more usually, within about 1% of one another. Accordingly, such compounds are particularly useful as detectable labels in sets of e-tag reagents in situations where the instrumentation employed cannot effectively resolve such compounds on a spectral level. The fact that such compounds have different charge and mass permits their use with one another as the detectable moiety component of the e-tag reagent with the added dimension of differing mobilities. Accordingly, the ability to multiplex determinations using e-tag reagents is expanded.

One aspect of the present invention is a set of electrophoretic tag (e-tag) probes for detecting the binding of or interaction between each or any of a plurality of ligands and one or more target antiligands. The set comprises j members, and each of said e-tag probes having the form:

(D, Mⱼ)—L—Tⱼ,

where

(a) D is a detection group comprising a detectable label;

(b) Tⱼ is a ligand capable of binding to or interacting with a target antiligand,

(c) L is a linking group connected to Tⱼ by a bond that is cleavable by a selected cleaving agent when the probe is bound to or interacting with the target antiligand, wherein cleavage by said agent produces an e-tag reporter of the form (D, Mⱼ)—L′, where L′ is the residue of L attached to (D, Mⱼ) after such cleavage,

(d) Mⱼ is a mobility modifier that imparts a unique and known electrophoretic mobility to a corresponding e-tag reporter of the form (D, Mⱼ)—L′, within a selected range of electrophoretic mobilities with respect to other e-tag reporters of the same form in the probe set; and

(e) (D, Mⱼ)— includes both D—Mⱼ— and Mⱼ—D—;

wherein at least two detectable labels are employed and are independently selected from compounds having substantially the same spectral properties and of the formula:

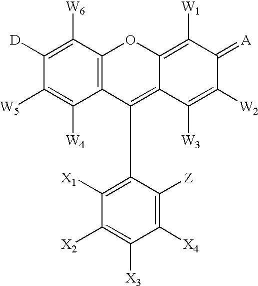

wherein:

Z is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, phosphorus containing species such as, e.g., phosphate, phosphite, and the like, polymer chains of from about 2 to about 10 monomer units such as, e.g. polyethylene glycol, polyamide, polyether, and the like,

A is O, N⁺(R¹)(R²) wherein R¹ and R² are independently H, lower alkyl, substituted lower alkyl, and the like,

D is OH, OR³ wherein R³ is lower alkyl, substituted lower alkyl, aryl, substituted aryl, and the like, N(R¹)(R²) wherein R¹ and R² are independently H, lower alkyl, substituted lower alkyl, and the like,

W¹, W², W³, W⁴, W⁵ and W⁶ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, and the like,

X¹-X⁴ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, and the like,

wherein W² and W³ may be taken together to form one or more rings comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like, and

wherein W⁴ and W⁵ may be taken together to form a ring comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like,

Lower alkenyl means a hydrocarbon similar to lower alkyl described above but having at least one carbon-carbon double bond and thus from 2 to 9 carbon atoms. Substituents on a lower alkenyl group may be as described above for substituted lower alkyl.

Lower alkynyl means a hydrocarbon similar to lower alkyl described above but having at least one carbon-carbon triple bond and thus from 2 to 9 carbon atoms. Substituents on a lower alkynyl group may be as described above for substituted lower alkyl.

In some instances one or more of the following provisos may apply:

the proviso that, when A is O, W⁵ is not phenyl, substituted phenyl, polycyclic aromatic, substituted polycyclic aromatic, heterocyclic, or substituted heterocyclic, and/or with the proviso that, when A is N(R¹)(R²) and none of W¹-W⁶ is carboxyl, X¹ and X⁴ are not chlorine, and/or

the proviso that, when A is O and one of W¹ or W² is lower alkyl or lower alkoxy, one of W⁵ or W⁶ is not hydrogen or halogen, and/or

the proviso that, when A is O, one of W², W⁵, X¹ or X⁴ is not chlorine, and/or

the proviso that, when A is O and one of W² or W⁵ is aliphatic hydrocarbylene, one of W¹, W³, W⁴ or W⁶ is not hydrogen, and/or

the proviso that, when Z is COOH and one of X^{1″}-X^{4″} is COOH, one of W1″-W^{6″} is other than hydrogen, and/or

the proviso that, when A is O and one of W¹ or W⁶ is alkoxy or thioalkyl, one of W³ or W⁴ is not hydrogen.

In one specific embodiment of the aforementioned probe set, at least two detectable labels are employed and are independently selected from compounds having substantially the same spectral properties and of the formula:

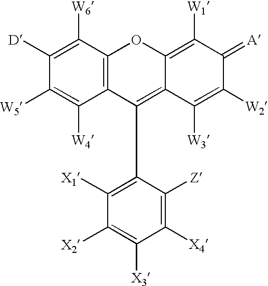

wherein:

Z′ is COOH,

A′ is O,

D′ is OH, OR^{3′} wherein R^{3′} is lower alkyl, substituted lower alkyl, aryl, substituted aryl,

W^{1′}, W^{2′}, W^{3′}, W^{4′} and W^{6′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, and the like,

W^{5′} is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, chlorine, fluorine, bromine, iodine, COOH, carboxylate, amide, nitrile, nitro, sulfonyl, sulfate, sulfone, amino, tethered amino, quaternary amino, imino, and the like,

X^{1′}-X^{4′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, and the like,

wherein W^{2′} and W^{3′} may be taken together to form one or more rings comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like, and

wherein W^{4′} and W^{5′} may be taken together to form a ring comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like.

In some instances one or more of the following provisos may apply:

the proviso that one of W^{2′}, W^{5′}, X^{1′} or X^{4′} is not chlorine, and/or

the proviso that, when one of W^{1′} or W^{2′} is lower alkyl or lower alkoxy, one of W^{5′} or W^{6′} is not hydrogen or halogen, and/or

the proviso that, when one of W^{2′} or W^{5′} is aliphatic hydrocarbylene, one of W^{1′}, W^{3′}, W^{4′} or W^{6′} is not hydrogen, and/or

the proviso that, when Z is COOH and one of X^{1″}-X^{4″} is COOH, one of W1″-W^{6″} is other than hydrogen, and/or

the proviso that, when one of W^{1′} or W^{6′} is alkoxy or thioalkyl, one of W^{3′} or W^{4′″} is not hydrogen.

In another specific embodiment of the above probe set, at least two detectable labels are employed and are independently selected from compounds having substantially the same spectral properties and of the formula:

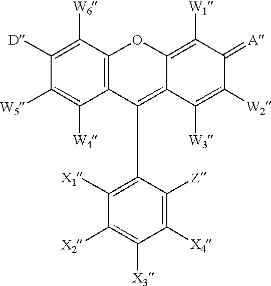

wherein

Z″ is COOH, and the like,

A″ is O, N(R^{1″})(R^{2″}) wherein R^{1″} and R^{2″} are independently lower alkyl, substituted lower alkyl, and the like,

D″ is OH, OR^{3″} wherein R^{3″} is lower alkyl, substituted lower alkyl, aryl, substituted aryl, and the like,

W^{1″} and W^{6″} are independently H, lower alkyl, substituted lower alkyl, COOH, chloro, fluoro, and the like,

W^{2″} and W^{5″} are independently H, lower alkyl, substituted lower alkyl, COOH, chloro, fluoro, and the like,

W^{3″} and W^{4″} are independently H, lower alkyl, substituted lower alkyl, COOH, chloro, fluoro, and the like,

wherein W^{2″} and W^{3″} may be taken together to form one or more rings comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like, and

wherein W^{4″} and W^{5″} may be taken together to form a ring comprising 4 to 14 atoms, preferably, 4 to 8 atoms, more preferably, 5 to 7 atoms, usually carbon atoms, and comprising 1 to 7 unsaturations, usually, 1 to 4 unsaturations, such as, e.g., benzo (from benzene), naptho (from naphthalene), anthro (from anthracene), and the like,

X^{1″}-X^{4″} are independently H, chloro, fluoro, COOH, bromo, iodo, and the like.

In some instances one or more of the following provisos may apply:

the proviso that, when A″ is N(R^{1″})(R^{2″}) and none of W^{1″}-W^{6″} is carboxyl, X^{1″} and X^{4″} are not chlorine, and/or

the proviso that, when A″ is O and W^{2″} and W^{3″} are taken together to form a benzo ring and W^{4″} and W^{5″} are not taken together to form a benzo ring, W^{5″} is H, halogen, lower alkyl, or COOH, and/or

the proviso that, when, A″ is O and X^{1″} and X^{4″} are chloro, one of W^{2″} or W^{5″} is not chloro, or when, A″ is O and W^{2″} and W^{5″} are chloro, one of X^{1″} or X^{4″} is not chloro, and/or

the proviso that, when Z is COOH and one of X^{1″}-X^{4″} is COOH, one of W1″-W^{6″} is other than hydrogen.

In another specific embodiment of the above probe set, the detectable labels are independently selected from compounds having substantially the same spectral properties and of the above formula wherein Z″ is carboxyl, W^{6″} and W^{1″} are lower alkyl, W^{5″} and W^{2″} are halogen, X^{2″} and X^{3″} are hydrogen or carboxyl and X^{1″} and X^{4″} are hydrogen or halogen.

In another specific embodiment of the above probe set, the detectable labels are independently selected from compounds having substantially the same spectral properties and of the above formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are hydrogen.

In another specific embodiment of the above probe set, the detectable labels are independently selected from compounds having substantially the same spectral properties and of the above formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are chloro.

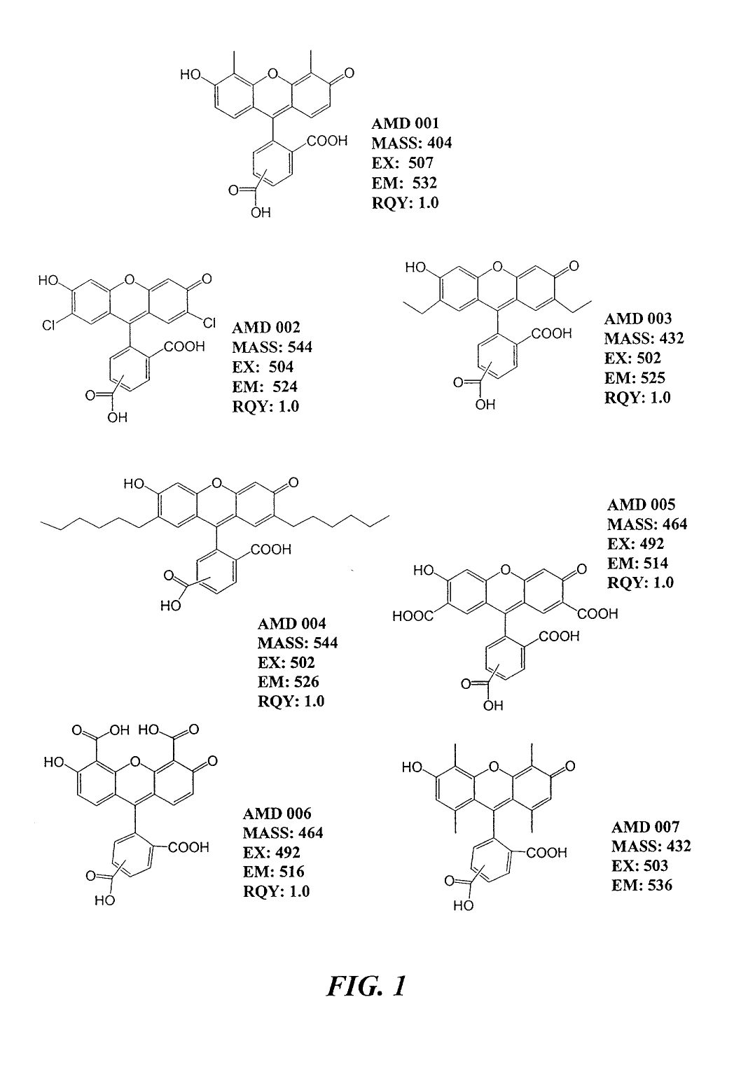

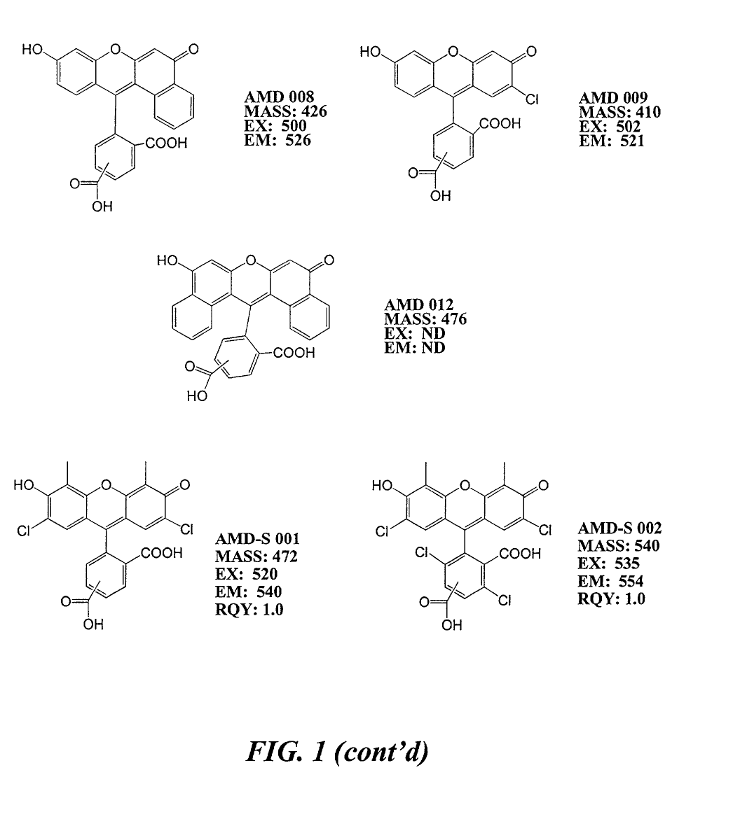

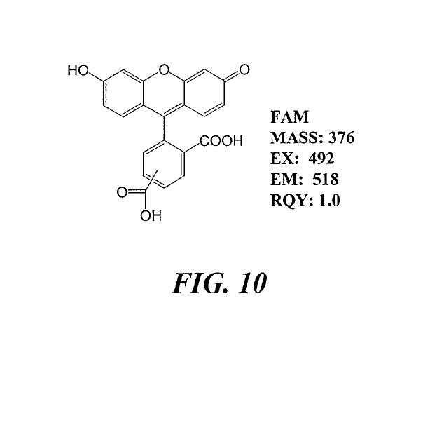

In another specific embodiment of the above probe set, the detectable labels are independently a compound of FIG. 1 having the same spectral properties as defined above. As can be seen, some of the compounds of FIG. 1, for example, compounds AMD 001 to AMD 013 as well as FAM (FIG. 10), have substantially the same spectral properties of excitation wavelength and emission wavelength but have different mass and charge and, thus, different mobilities.

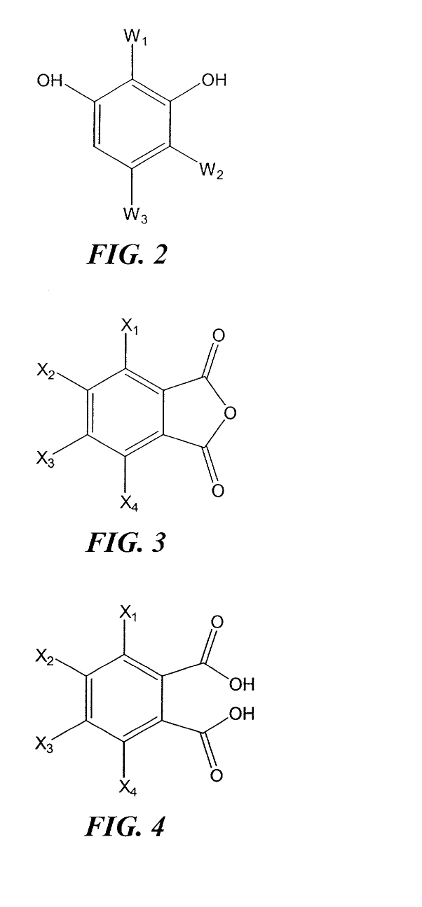

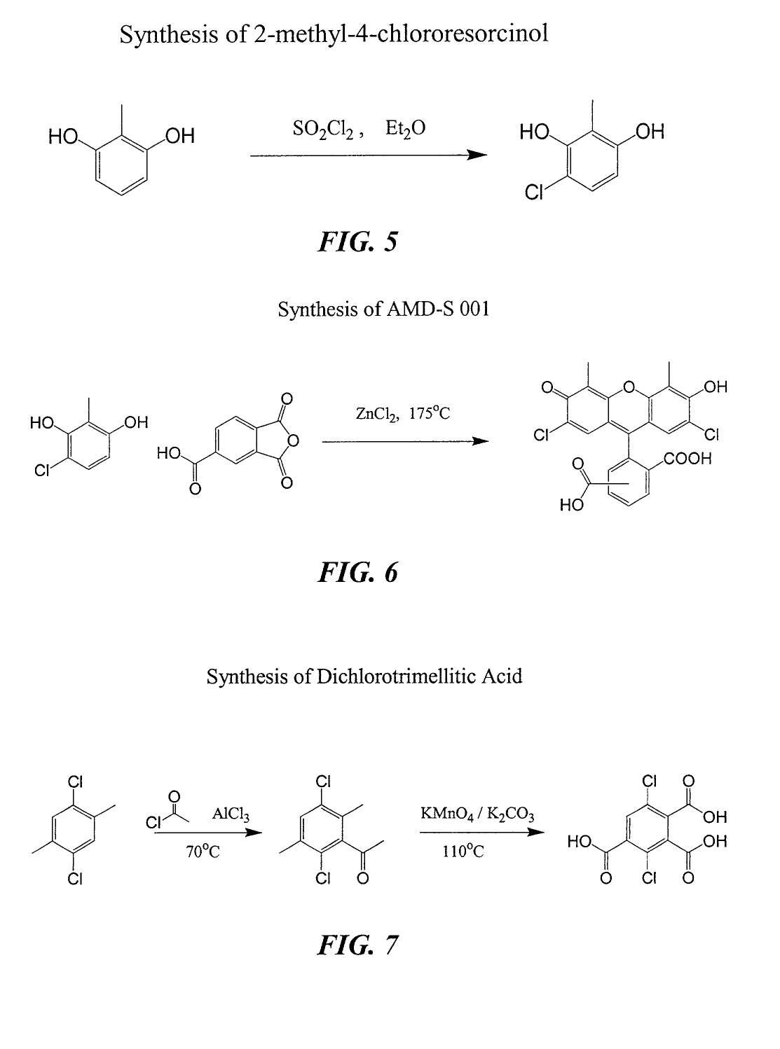

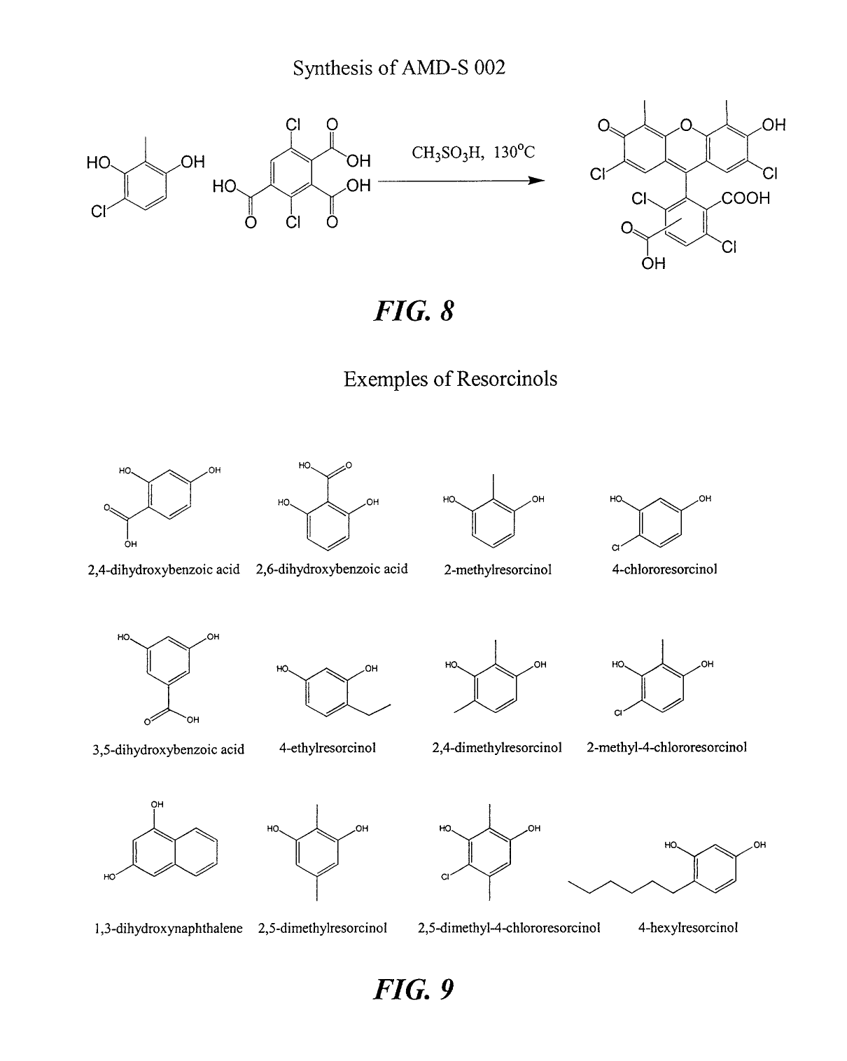

The aforementioned fluorescent compounds may be synthesized in a number of different synthetic approaches such as those represented in FIGS. 5-8. These approaches generally involve the reaction of an appropriate resorcinol, as generally represented in FIG. 2, with an appropriate phthalic acid anhydride, as generally represented in FIG. 3, or an appropriate phthalic acid, as generally represented in FIG. 4, with heating in the presence of a suitable condensation catalyst. In the approach of FIG. 6, an example of the condensation reaction is carried out at elevated temperature of about 150 to about 200° C., usually, about 175° C. A condensation catalyst is employed such as, for example, zinc chloride, aluminum chloride, and the like. In the example in the approach of FIG. 8, the appropriate resorcinol and phthalic acid anhydride are heated at an elevated temperature, usually, about 100 to about 150° C., more usually, about 130° C. The catalyst for condensation in this approach may be any acid suitable for condensation reactions of this type such as, for example, methane sulfonic acid, tosic acid, and the like. By choosing the appropriately substituted resorcinol and phthalic acid anhydride, the aforementioned fluorescent compounds may be synthesized. Examples of resorcinols employed to prepare the aforementioned fluorescent compounds are set forth in FIG. 9.

The present methods may be used to synthesize both symmetrical and unsymmetrical fluorescent compounds as well as various regioisomers. The synthesis of unsymmetrical fluorescein derivatives employs a benzophenone intermediate that is a decomposition product of a carboxy fluorescein synthesized from an acid anhydride and the first of two different resorcinols. The benzophenone is subsequently reacted with the second of the two different resorcinols, under conditions that are similar to or identical to those described above for the symmetrical fluorescein derivatives, to generate the desired material. Isolation is also as described for the symmetrical fluorescein derivatives.

In another aspect of the present invention, the electrophoretic tags have a linker that provides linkage between the detectable label comprising a mobility modifier and the target binding moiety. The detectable label is the same for all e-tags and the mobility modifiers are different for each of the e-tags. It should be noted that the detectable label may be fluorescein or any of the above compounds set forth above.

In accordance with this aspect of the invention, an e-tag probe for use in electrophoresis comprises a substituted luminescent compound, usually a fluorescent compound, attached to a target-binding moiety by a cleavable linkage directly to the luminescent compound. In a preferred embodiment an e-tag probe may be represented by the formula:

M—D—L—T

wherein D is a label such as, for example, a fluorescer, M is a mobility-modifying moiety, L is a bond or a linking group linking D and T and comprising a cleavable linkage usually at the point of attachment to D so that, upon cleavage, an e-tag reporter consists essentially of M—D, and T comprises a target-binding region. The attachment of T to L is dependent on the nature of the target and the target-binding region. Where the target-binding region is a polynucleotide, T may be attached to L at a nucleoside base, purine or pyrimidine, naturally occurring or synthetic, or other functionality that may serve to participate in the synthesis of an oligomer. Where the target-binding region is a poly(amino acid), T may be attached to L at an amino acid, either naturally occurring or synthetic, or other functionality that may serve to participate in the synthesis of a poly(amino acid). M provides a major factor in the differences in mobility between the different e-tag reporters, M—D. The aforementioned e-tag probes are designed such that cleavage of the cleavable linkage results in e-tag reporters that consist essentially of M—D wherein D is the same fluorescer in each of the e-tag reporters and M differs from one e-tag reporter to another. In some circumstances, a less preferred embodiment may be employed wherein the cleavable linkage is positioned in L such that a portion L′ of L is included with e-tag reporter M—D—L′. In this situation, the portion of L included with each e-tag reporter is the same, that is, L′ is common among the e-tag reporters in a set of e-tag reporters.

In one representation of the invention, M has been substantially described as the mobility-modifying moiety and as indicated previously may include charged groups, uncharged polar groups or be non-polar. The groups may be alkylene and substituted alkylenes, oxyalkylene and polyoxyalkylene, particularly alkylene of from about 2 to about 3 carbon atoms, arylenes and substituted arylenes, polyamides, polyethers, polyalkylene amines, etc. Substituents may include heteroatoms, such as halo, phosphorous, nitrogen, oxygen, sulfur, etc., where the substituent may be halo, nitro, cyano, non-oxo-carbonyl, e.g., ester, acid and amide, oxo-carbonyl, e.g., aldehyde and keto, amidine, urea, urethane, guanidine, carbamyl, amino and substituted amino, particularly alkyl substituted amino, azo, oxy, e.g., hydroxyl and ether, etc., where the substituents are generally of from about 0 to about 10 carbon atoms, while L are generally of from about 1 to about 100 carbon atoms, more usually of from about 1 to about 60 carbon atoms and preferably about 1 to about 36 carbon atoms.

M may be joined to the label by any convenient functionality, such as carboxy, amino, oxy, phospho, thio, iminoether, etc., where in many cases the detection group or label and the mobility modifier have a convenient functionality for linkage. The important aspect of the linkage between the mobility modifier and the detection group, unlike for L, is that the former does not include a cleavable portion or linker so that an e-tag reporter comprising the label and a mobility-modifying moiety be released upon cleavage of the cleavable linkage in L. For the most part, the linker may be a bond, where the label is directly bonded to the mobility modifier, or a linking group. Usually, the mobility modifier is bound to the label by a bond.

The number of heteroatoms in M is sufficient to impart the desired mobility and/or charge to the label conjugate, usually from about 1 to about 200, more usually from about 2 to about 100, heteroatoms. The heteroatoms in M may be substituted with atoms other than hydrogen.

In one embodiment of the present invention the label conjugates having different charge-to-mass ratios may comprise fluorescent compounds, each of which are linked to molecules that impart a charge to the released fluorescent compound-mobility modifier conjugate. As indicated previously, desirably the mobility modifier has an overall negative charge, preferably having in the case of a plurality of groups, groups of the same charge, where the total charge may be reduced by having one or more oppositely charged moieties.

In one embodiment the mobility modifiers may be oligomers, where the monomers may differ as to mass and charge. For convenience and economy, monomers will generally be commercially available, but if desired, they may be synthesized. Monomers which are commercially available and readily lend themselves to oligomerization include amino acids, both natural and synthetic, nucleotides, both natural and synthetic, and monosaccharides, both natural and synthetic, while other monomers include hydroxyacids, where the acids may be organic or inorganic, e.g., carboxylic, phosphoric, boric, sulfonic, etc., and amino acids, where the acid is inorganic, and the like. In some instances, nucleotides, natural or synthetic, may find use. The monomers may be neutral, negatively charged or positively charged. The charges of the monomers in the mobility modifiers may be the same so that reference to the charge-to-mass ratio is related to the same charge. The label may have a different charge from the mobility modifier or mobility-modifying moiety. Such a situation is treated as if the number of charges is reduced by the number of charges on the mobility-modifying moiety. For natural amino acids, the positive charges may be obtained from lysine, arginine and histidine, while the negative charges may be obtained from aspartic and glutamic acid. For nucleotides, the charges will be obtained from the phosphate and any substituents that may be present or introduced onto the base. For sugars, sialic acid, uronic acids of the various sugars, or substituted sugars may be employed.

The charge-imparting moieties of M may be, for example, amino acids, tetraalkylammonium, phosphonium, phosphate diesters, carboxylic acids, thioacids, sulfonic acids, sulfate groups, phosphate monoesters, and the like and combinations of one or more of the above. The number of the above components of M is such as to achieve the desired number of different charge-imparting moieties. The amino acids may be, for example, lysine, aspartic acid, alanine, gamma-aminobutyric acid, glycine, β-alanine, cysteine, glutamic acid, homocysteine, β-alanine and the like. The phosphate diesters include, for example, dimethyl phosphate diester, ethylene glycol linked phosphate diester, and so forth. The thioacids include, by way of example, thioacetic acid, thiopropionic acid, thiobutyric acid and so forth. The carboxylic acids preferably have from about 1 to about 30 carbon atoms, more preferably, from about 2 to about 15 carbon atoms and preferably comprise one or more heteroatoms and may be, for example, acetic acid derivatives, formic acid derivatives, succinic acid derivatives, citric acid derivatives, phytic acid derivatives and the like.

In one approach M may have two sub-regions, a common charged sub-region, which are common to a group of e-tag moieties, and a varying uncharged, a non-polar or polar sub-region, that varies the charge-to-mass ratio. This permits ease of synthesis, provides for relatively common chemical and physical properties and permits ease of handling. For negative charges, one may use dibasic acids that are substituted with functionalities that permit low orders of oligomerization, such as hydroxy and amino, where amino will usually be present as neutral amide. These charge-imparting groups provide aqueous solubility and allow for various levels of hydrophobicity in the other sub-region. Thus, the uncharged sub-region could employ substituted dihydroxybenzenes, diaminobenzenes, or aminophenols, with one or greater number of aromatic rings, fused or non-fused, where substituents may be halo, nitro, cyano, alkyl, etc., allowing for great variation in molecular weight by using a common building block. Where the other regions of the e-tag moiety impart charge to the e-tag reporter, M may be neutral.

Conjugates of particular interest comprise a fluorescer and a different alkylene chain, alkylene oxide chain, alkyleneamine chain, amino acid or combinations thereof in the form of a peptide or combinations of amino acids and thioacids or other carboxylic acids. Such compounds are represented by the formula:

M′—D′—T′—

wherein D′ is a fluorescer, M′ is an alkylene chain, alkylene oxide chain, an amino acid or a peptide or combinations of amino acids and thioacids or other carboxylic acids and T′ is a target-binding moiety.

In a particular embodiment the label conjugates may be represented by the formula:

(M″)ₙ-Fluorescer-L^{a}—T″

wherein M″ is an alkylene chain, an alkylene oxide chain, an amino acid chain, L^{a} is a bond or a linking group of from 1 to 20 atoms other than hydrogen and comprising a cleavable linkage, n is 1 to 20, and T″ is a target binding moiety. In this embodiment T is linked to the Fluorescer by a linking group having a cleavable linkage.

An example of label conjugates in the above embodiment, by way of illustration and not limitation, may be represented by the formula wherein Fl is Fluorescer and dN is a deoxynucleotide, e.g., dT, dC, dU, dG or dA, N is a nucleotide, e.g., T, C, U, G, or A, M′″ is an alkylene oxide chain and L^{b} is an alkylene oxide chain:

M′″-dN(Fl)-L^{b}—N

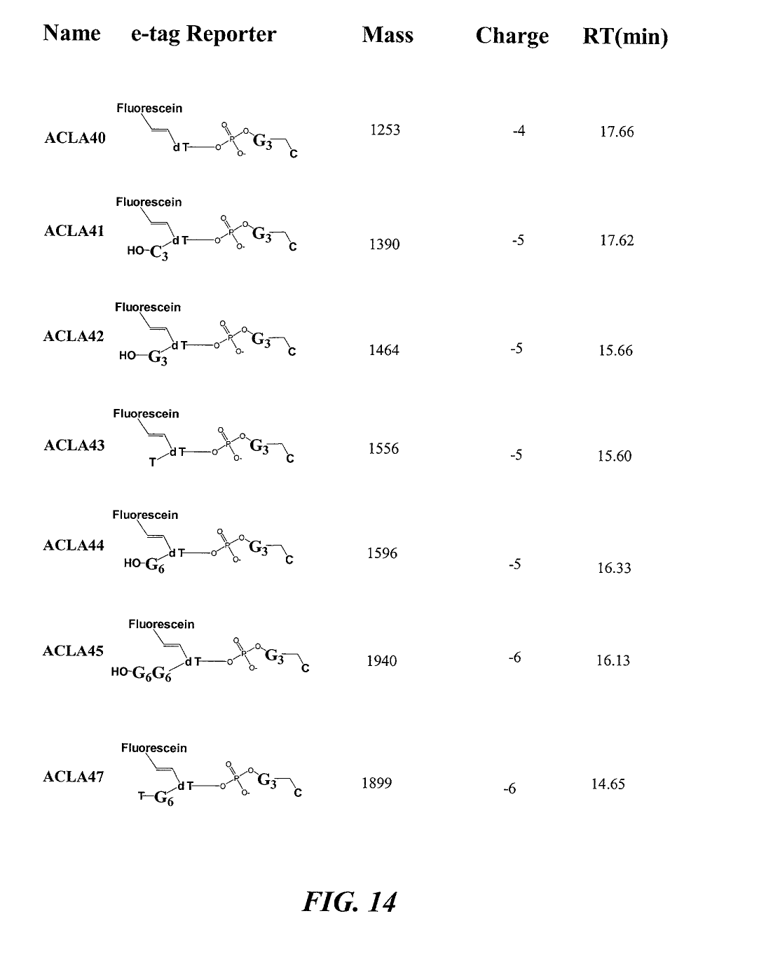

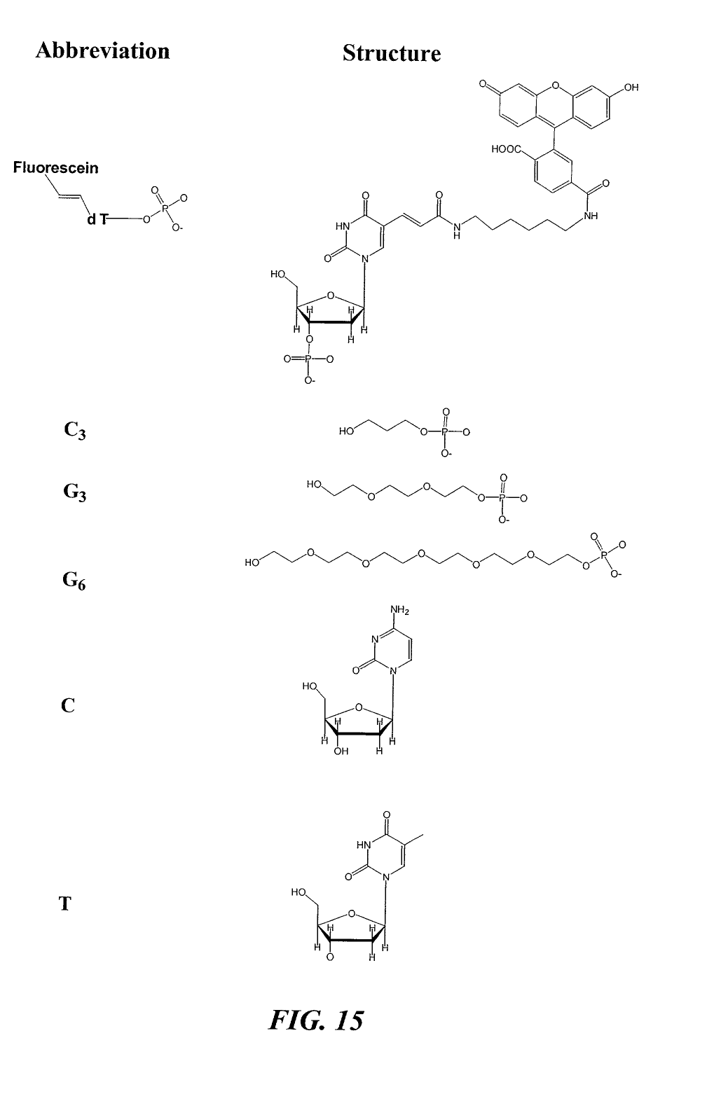

Particular examples of compounds of the above formula are set forth in FIGS. 14 and 15. It should be noted that the structures in the abbreviations in FIG. 15 would include an appropriate protecting group such as DMT or other substituent when intended to represent the components for the synthesis of the compounds of FIG. 14. Likewise, the structures in the abbreviations in FIG. 15 would not include an appropriate atom such as H when intended to represent the corresponding part of the structure of the compounds of FIG. 14.

The e-tag probes in the aforementioned embodiment may be prepared by phosphoramidite coupling methods well known in the art. The extendable fluorescein derivative used in the synthesis is available from Glen Research, Sterling Va. The electrophoretic conditions employed are set forth below in the section entitled “Analysis of Reaction Products.”

Phosphoramidite derivatives of the present fluorescent compounds may be synthesized by methods that are well-known in the art. Briefly, phenolic hydroxyls of the fluorescent compounds are protected with suitable protecting groups that can be removed with a deprotection agent employed in the polynucleotide synthesis such as, for example, ammonia, ethanolamine, methylamine/ammonium hydroxide, and so forth. The protecting groups include by way of illustration and not limitation esters of benzoic acid, esters of pivalic acid, and the like. A linking moiety of the fluorescent compound such as a carboxyl group is activated with a suitable activating agent such as, for example, carbodiimide, N-hydroxysuccinimide, and so forth. The activated linking moiety is reacted with an alcohol linker such as, e.g., an aminoalcohol, and the like, to give the protected fluorescent compound with a free alcohol group. The resultant compound with free alcohol functionality is reacted with a phosphitylating agent using standard procedures.

The aforementioned compounds may be used to prepare phosphoramidite reagents for use in the synthesis of polynucleotides and the like. Such reagents are particularly useful for the automated synthesis of labeled polynucleotides comprising one or more labels of the invention. The labeled phosphoramidite reagents may be reacted with a 5′-hydroxyl group of a nucleotide or polynucleotide to form a phosphite ester, which is oxidized to give a phosphate ester. The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643 and elsewhere. The phosphoramidite and phosphite triester approaches are most broadly used, but other approaches include the phosphodiester approach, the phosphotriester approach and the H-phosphonate approach.

It is within the purview of the present invention to use the aforementioned e-tag probes in conjunction with e-tag probes employing other types of labels. In this regard several sets of e-tag probes may be employed in a multiplexed assay. One set of e-tag probes may comprise the aforementioned probes and another set of e-tag probes may comprise labels that have differing spectral properties.

B. Electrophoretic Tags for Use in Electrophoresis

The electrophoretic tag, which is detected, will comprise the mobility modifier, generally a label, and optionally a portion of the target-binding moiety, all of the target-binding moiety when the target is an enzyme and the target-binding moiety is the substrate. Generally, the electrophoretic tag will have a charge/mass ratio in the range of about −0.0001 to 0.1, usually in the range of about −0.001 to about 0.5. Mobility is q/M^{⅔}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more.

In those instances where a label is not present on the e-tag bound to the target-binding moiety (e.g., a snp detection sequence), the mixture may be added to a functionalized fluorescent tag bearing a mobility modifier to label the e-tag with a fluorescer-mobility modifier. For example, where a thiol group is present, the fluorescer could have an activated ethylene, such as maleic acid to form the thioether. For hydroxyl groups, one could use activated halogen or pseudohalogen for forming an ether, such as an a-haloketone. For carboxyl groups, carbodiimide and appropriate amines or alcohols would form amides and esters, respectively. For an amine, one could use activated carboxylic acids, aldehydes under reducing conditions, activated halogen or pseudohalogen, etc. When synthesizing oligopeptides, protective groups are used. These could be retained while the fluorescent moiety is attached to an available functionality on the oligopeptide.

D. E-tag Reagents—Synthesis

The e-tag reagents may be prepared utilizing conjugating techniques that are well known in the art. The mobility modifier may be synthesized from smaller molecules that have functional groups that provide for linking of the molecules to one another, usually in a linear chain. Such functional groups include carboxylic acids, amines, and hydroxy- or thiol-groups. In accordance with one embodiment of the present invention, the mobility modifier may have one or more side groups pending from the core chain. The side groups have a functionality to provide for linking to a label or to another molecule of the mobility modifier.

Common functionalities resulting from the reaction of the functional groups employed are exemplified by forming a covalent bond between the molecules to be conjugated. Such functionalities are disulfide, amide, thioamide, dithiol, ether, urea, thiourea, guanidine, azo, thioether, carboxylate and esters and amides containing sulfur and phosphorus such as, e.g. sulfonate, phosphate esters, sulfonamides, thioesters, etc., and the like.

Illustrative of the synthesis for a polyalkylene chain for the mobility modifier is the employment of a diol, such as an alkylene diol, polyalkylene diol, with alkylene of from 2 to 3 carbon atoms, alkylene amine or poly(alkylene amine) diol, where the alkylenes are of from 2 to 3 carbon atoms and the nitrogens are substituted, for example with blocking groups or alkyl groups of from 1-6 carbon atoms, where one diol is blocked with a conventional protecting group, such as a dimethyltrityl group. This group can serve as the mass-modifying region and with the amino groups as the charge-modifying region as well. If desired, the mass modifier can be assembled using building blocks that are joined through phosphoramidite chemistry. In this way the charge modifier can be interspersed within the mass modifier. For example, one could prepare a series of polyethylene oxide molecules having 1, 2, 3 . . . n units. Where it is desired to introduce a number of negative charges, a small polyethylene oxide unit may be used and the mass and charge-modifying region may be built by having a plurality of the polyethylene oxide units joined by phosphate units. Alternatively, by employing a large spacer, fewer phosphate groups would be present, so that without large mass differences, large differences in mass-to-charge ratios are obtained. The chemistry for performing the types of syntheses to form the charge-imparting moiety or mobility modifier is well known in the art.

For synthesis of peptide chains, see Marglin, et al., Ann. Rev. Biochem. (1970) 39:841-866. In general, such syntheses involve blocking, with an appropriate protecting group, those functional groups that are not to be involved in the reaction. The free functional groups are then reacted to form the desired linkages. The peptide can be produced on a resin as in the Merrifield synthesis (Merrifield, J. Am. Chem. Soc. (1980) 85:2149-2154 and Houghten et al., Int. J. Pep. Prot. Res. (1980) 16:311-320. The peptide is then removed from the resin according to known techniques.

A summary of the many techniques available for the synthesis of peptides may be found in J. M. Stewart, et al., “Solid Phase Peptide Synthesis, W. H. Freeman Co, San Francisco (1969); and J. Meienhofer, “Hormonal Proteins and Peptides”, (1973), vol. 2, p 46, Academic Press (New York), for solid phase peptide synthesis; and E. Schroder, et al., “The Peptides, vol. 1, Academic Press (New York), 1965 for solution synthesis.

In general, these methods comprise the sequential addition of one or more amino acids, or suitably protected amino acids, to a growing peptide chain. Normally, a suitable protecting group protects either the amino or carboxyl group of the first amino acid. The protected or derivatized amino acid can then be either attached to an inert solid support or utilized in solution by adding the next amino acid in the sequence having the complementary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide. The protecting groups are removed, as desired, according to known methods depending on the particular protecting group utilized. For example, the protecting group may be removed by reduction with hydrogen and palladium on charcoal, sodium in liquid ammonia, etc.; hydrolysis with trifluoroacetic acid, hydrofluoric acid, and the like.

In one exemplary approach, after the synthesis of the peptide is complete, the peptide is removed from the resin by conventional means such as ammonolysis, acidolysis and the like. The fully deprotected peptide may then be purified by techniques known in the art such as chromatography, for example, adsorption chromatography, ion exchange chromatography, partition chromatography, high performance liquid chromatography, thin layer chromatography, and so forth.

As can be seen, the selected peptide representing a charge-imparting moiety may be synthesized separately and then attached to the label either directly or by means of a linking group, which is different from the linking group involved in the attachment of the label to the target binding moiety. On the other hand, the peptide may be synthesized as a growing chain on the label. In any of the above approaches, the linking of the peptide or amino acid to the label may be carried out using one or more of the techniques described above for the synthesis of peptides or for linking moieties to labels.

Synthesis of e-tags comprising nucleotides can be easily and effectively achieved via assembly on a solid phase support during probe synthesis, using standard phosphoramidite chemistries. In one approach, the e-tag probe is constructed sequentially from a single or several monomeric phosphoramidite building blocks (one containing a dye residue), which are chosen to generate tags with unique electrophoretic mobilities based on their mass to charge ratio. The e-tag probe is thus composed of monomeric units of variable charge to mass ratios bridged by phosphate linkers.

The aforementioned label conjugates with different electrophoretic mobility permit a multiplexed amplification and detection of multiple targets, e.g. nucleic acid targets, protein targets and so forth. The label conjugates are linked to target binding moieties such as, e.g., oligonucleotides, in a manner similar to that for labels in general, by means of linkages that are enzymatically cleavable. It is, of course, within the purview of the present invention to prepare any number of label conjugates for performing multiplexed determinations. Accordingly, for example, with 40 to 50 different label conjugates separated in a single separation channel and 96 different amplification reactions with 96 separation channels on a single plastic chip, one can detect 4000 to 5000 single nucleotide polymorphisms.

The e-tag may be assembled having an appropriate functionality on the label for linking to the target binding moiety. Thus, for oligonucleotides as the target binding moieties, a phosphoramidite or phosphate ester at the linking site may be used to bond to an oligonucleotide chain, either 5′ or 3′, particularly after the oligonucleotide has been synthesized, while still on a solid support and before the blocking groups have been removed. While other techniques exist for linking the oligonucleotide to the label of the e-tag, such as having a functionality at the oligonucleotide terminus that specifically reacts with a functionality on the label of the e-tag, such as maleimide and thiol, or amino and carboxy, or amino and keto under reductive animation conditions, the phosphoramidite addition is preferred. For a peptide as the target binding moiety, a variety of functionalities can be employed, much as with the oligonucleotide functionality, although phosphoramidite chemistry may only occasionally be appropriate. Thus, the functionalities normally present in a peptide, such as carboxy, amino, hydroxy and thiol may be the targets of a reactive functionality for forming a covalent bond.

Of particular interest in preparing e-tag labeled nucleic acid target binding moieties (e-tag probes) is using the solid support phosphoramidite chemistry to build the e-tag as part of the oligonucleotide synthesis. Using this procedure, the next succeeding phosphate is attached at the 5′ or 3′ position, usually the 5′ position of the oligonucleotide chain. The added phosphoramidite may have a natural nucleotide or an unnatural nucleotide. Instead of phosphoramidite chemistry, other types of linkers may be used, such as thio analogs, amino acid analogs, etc. The chemistry that is employed is the conventional chemistry used in oligonucleotide synthesis, where building blocks other than nucleotides are used, but the reaction is the conventional phosphoramidite chemistry and the blocking group is the conventional dimethoxytrityl group. Of course, other chemistries compatible with automated synthesizers can also be used, but there is no reason to add additional complexity to the process.

For peptides, the e-tags will be linked in accordance with the chemistry of the label, the linking group and the availability of functionalities on the peptide target binding moiety. For example, with Fab′ fragments specific for a target compound, a thiol group will be available for using an active olefin, e.g. maleimide, for thioether formation. Where lysines are available, one may use activated esters capable of reacting in water, such as nitrophenyl esters or pentafluorophenyl esters, or mixed anhydrides as with carbodiimide and half-ester carbonic acid. There is ample chemistry for conjugation in the literature, so that for each specific situation, there is ample precedent in the literature for the conjugation.

For separations based on sorption, adsorption and/or absorption, the nature of the e-tag reporters to provide for differentiation can be relatively simple. By using differences in composition, such as aliphatic compounds, aromatic compounds and halo derivatives thereof, the determinations may be made with gas chromatography, with electron capture or negative ion mass spectrometry, when electronegative atoms are present. In this way hydrocarbons or halo-substituted hydrocarbons may be employed as the e-tag reporters bonded to a releasable linker. See, U.S. Pat. Nos. 5,565,324 and 6,001,579, which are specifically incorporated by reference as to the relevant disclosure concerning cleavable groups and detectable groups.

E. Sets of e-tags

In another embodiment the invention concerns libraries comprising sets of electrophoretic tag (e-tag) probes for detecting the binding of or interaction between each or any of a plurality of ligands and one or more target antiligands. The set comprises j members, and each of the e-tag probes has the form: Mⱼ—D—L—Tⱼ, wherein (a) D is a detection group comprising a detectable label that is the same for all of the e-tag probes; (b) Tⱼ is a ligand capable of binding to or interacting with a target antiligand, (c) L is a bond or a linking group linking D and Tⱼ and comprising a cleavable linkage at the point of attachment to D or within L at a point that is common to all of the e-tag probes, wherein cleavage of the cleavable linkage produces an e-tag reporter of the form Mⱼ—D or Mⱼ—D—L′, where L′ is the residue of L attached to Mⱼ—D after such cleavage, and d) Mⱼ is a mobility modifier having a charge/mass ratio or a mass that imparts a unique and known electrophoretic mobility to a corresponding e-tag reporter, within a selected range of electrophoretic mobilities with respect to other e-tag reporters of the same form in the probe set.

The libraries will ordinarily have at least about 5 members, usually at least about 10 members, and may have 100 members or more, for convenience generally having about 50-75 members. Some members may be combined in a single container or be provided in individual containers where permitted. The members of the library will be selected to provide clean separations in electrophoresis, when capillary electrophoresis is the analytical method. To that extent, mobilities will differ as described above, where the separations may be greater, the larger the larger the number of molecules in the band to be analyzed. Particularly, non-sieving media may be employed in the separation.

Depending upon the reagent to which the e-tag is attached, there may be a single e-tag or a plurality of e-tags, generally ranging from about 1 to about 100, more usually ranging from about 1 to about 40, more particularly ranging from about 1 to about 20. The number of e-tags bonded to a single target-binding moiety will depend upon the sensitivity required, the solubility of the e-tag conjugate, the effect on the assay of a plurality of e-tags, and the like. For oligomers or polymers, such as nucleic acids and poly(amino acids), e.g. peptides and proteins, one may have one or a plurality of e-tags, while for synthetic or naturally occurring non-oligomeric compounds, usually there will be only 1 to about 3, more usually 1 to about 2 e-tags.

For 20 different e-tag reporters, only 5 different mass-modifying regions, one phosphate link and four different detectable regions are required. For 120 e-tag reporters, only 10 different mass-modifying regions, 3 different charge-modifying regions and 4 different detectable regions are needed. For 500 different e-tag reporters, only 25 different mass-modifying regions, 5 different charge-modifying regions and 4 different detectable regions are needed.

III. Methods for Use of the e-tags

The methodologies that may be employed involve heterogeneous and homogeneous techniques, where heterogeneous normally involves a separation step, where unbound label is separated from bound label, where homogeneous assays do not require, but may employ, a separation step. One group of assays will involve nucleic acid detection, which includes sequence recognition, snp detection and scoring, transcription analysis, allele determinations, HLA determinations, or other determination associated with variations in sequence. The use of the determination may be forensic, mRNA determinations, mutation determinations, allele determinations, MHC determinations, haplotype determinations, single nucleotide polymorphism determinations, etc. The methodology may include assays dependent on 5′-nuclease activity, as in the use of the polymerase chain reaction or in Invader technology, 3 ′-nuclease activity, restriction enzymes, or ribonuclease H. All of these methods involving catalytic cleavage of a phosphate linkage, where one to two oligonucleotides are bound to the target template.

In addition, the subject heterogeneous assays require that the unbound labeled reagent be separable from the bound labeled reagent. This can be achieved in a variety of ways. Each way requires that a reagent bound to a solid support that distinguishes between the complex of labeled reagent and target. The solid support may be a vessel wall, e.g. microtiter well plate well, capillary, plate, slide, beads, including magnetic beads, liposomes, or the like. The primary characteristics of the solid support is that it permits segregation of the bound labeled specific binding member from unbound probe, and that the support does not interfere with the formation of the binding complex, nor the other operations of the determination.

The solid support may have the complex directly or indirectly bound to the support. For directly bound, the binding member or e-tag probe is covalently or non-covalently bound to the support. For proteins, many surfaces provide non-diffusible binding of a protein to the support, so that the protein is added to the support and the protein is allowed to bind; weakly bound protein is washed away and an innocuous protein is added to coat any actively binding areas that are still available. The surface may be activated with various functionalities that form covalent bonds with a binding member. These groups may include imino halides, activated carboxyl groups, e.g. mixed anhydrides or acyl halides, amino groups, α-halo or pseudohaloketones, etc. The specific binding member bound to the surface of the support may be any molecule that permits the binding portion of the molecule, e.g. epitope, to be available for binding by the reciprocal member. Where the binding member is polyepitopic, e.g. proteins, this is usually less of a problem, since the protein will be polyepitopic and even with random binding of the protein to the surface, the desired epitope will be available for most of the bound molecules. For smaller molecules, particularly under 5 kDal, an active functionality may be present on the specific binding member that preserves the binding site, where the active functionality reacts with a functionality on the surface of the support. The same functionalities described above may find use. Conveniently, one may use the same site for preparing the conjugate immunogen to produce antibodies as the site for the active functionality for linking to the surface.

Instead of nucleic acid pairing, one may employ specific binding member pairing. There are a large number of specific binding pairs associated with receptors, such as antibodies, poly- and monoclonal, enzymes, surface membrane receptors, lectins, etc., and ligands for the receptors, which may be naturally occurring or synthetic molecules, protein or non-protein, such as drugs, hormones, enzymes, ligands, etc. The specific binding pair has many similarities to the binding of homologous nucleic acids, significant differences being that one normally cannot cycle between the target and the agent and one does not have convenient phosphate bonds to cleave. For heterogeneous assays, the binding of the specific binding pair is employed to separate the bound from the unbound e-tag bonded agents, while with homogeneous assays, the proximity of the specific binding pairs allow for release of the e-tags from the complex. For an inclusive but not exclusive listing of the various manners in which the subject invention may be used, Tables 1 and 2 are provided.

Once the target binding moiety conjugated with the e-tag has been prepared, it may be used in a number of different assays. The samples may be processed using lysis, nucleic acid separation from proteins and lipids and vice versa, and enrichment of different fractions. For nucleic acid related determinations, the source of the DNA may be any organism, prokaryotic and eukaryotic cells, tissue, environmental samples, etc. The DNA or RNA may be isolated by conventional means, RNA may be reverse transcribed, DNA may be amplified, as with PCR, primers may be used with capture ligands for use in subsequent processing, the DNA may be fragmented using restriction enzymes, specific sequences may be concentrated or removed using homologous sequences bound to a support, or the like. Proteins may be isolated using precipitation, extraction, and chromatography. The proteins may be present as individual proteins or combined in various aggregations, such as organelles, cells, viruses, etc. Once the target components have been preliminarily treated, the sample may then be combined with the e-tag reporter targeted binding proteins.

For a nucleic acid sample, after processing, the probe mixture of e-tags for the target sequences is combined with the sample under hybridization conditions, in conjunction with other reagents, as necessary. Where the reaction is heterogeneous, the target-binding sequence has a capture ligand for binding to a reciprocal binding member for sequestering hybrids to which the e-tag probe is bound. In this case, all of the DNA sample carrying the capture ligand is sequestered, both with and without e-tag reporter labeled probe. After sequestering the sample, non-specifically bound e-tag reporter labeled probe is removed under a predetermined stringency based on the probe sequence, using washing at an elevated temperature, salt concentration, organic solvent, etc. Then, the e-tag reporter is released into an electrophoretic buffer solution for analysis.

As indicated in Table 1, for amplification one may use thermal cycling. Tables 1 and 2 indicate the properties of binding assays (solution phase e-tag generation followed by separation by CE, HPLC or mass spectra) and multiplexed assays (2-1000) leading to release of a library of e-tags, where every e-tag codes for a unique binding event or assay.

The cleavage of the nucleic acid bound to the template results in a change in the melting temperature of the e-tag residue with release of the e-tag. By appropriate choice of the primer and/or protocol, one can retain the primer bound to the template and the e-tag containing sequence can be cleaved and released from the template to be replaced by an e-tag containing probe.

TABLE 1
Binding and Multiplexed Assays.

| Formats | Recognition Event | Amplification Mode | e-tag Release |
|---|---|---|---|
| Multiplexed assays | Solution hybridization | PCR, Invader | 5′ nuclease |
| Sequence | followed by enzyme recognition | | 3′ nuclease |
| recognition for | | | Restriction |
| example for | | | enzyme |
| multiplexed gene | | | Ribonuclease H |
| expression, SNP's | Solution hybridization | Amplification due to | Singlet Oxygen |
| scoring etc. . . | followed by channeling | turnover of e-tag binding | (′O₂) |
| | | moiety; OR | |
| | | amplification due to release | Hydrogen |
| | | of multiple e-tags (10 to | Peroxide (H₂O₂) |
| | | 100,000) per binding event | Light, |
| | | | energy transfer |
| Patches in | Target captured on solid surface; | Amplification from release | Light, enzyme, |
| microfluidic | e-tag probe mixture hybridized to | of multiple e-tag reporters | ′O₂, |
| channels - | target; unbound probes removed; | (10 to 100,000) per probe | H₂O₂, Fluoride, |
| integrated assay and | e-tag reporter is released, | | reducing agent, |
| separation | separated and identified. | | MS others |
| device | | | |

TABLE 2
Immunoassays

| Format | Recognition Event | Amplification Mode | e-tag Release |
|---|---|---|---|
| Proteomics | Sandwich assays | A few (2-10) e-tags | Singlet Oxygen |
| Multiplexed | Antibody-1 decorated with | released per binding event | (′O₂) |
| Immunoassays | Sensitizer while antibody-2 is | | |
| | decorated with singlet oxygen | OR | |
| | cleavable e-tags | | |
| | Competition assays | Amplification from | |
| | Antibody-1 decorated with | release of multiple | |
| | Sensitizer while antibody-2 is | e-tags (10 to 100,000) per | |
| | decorated with singlet oxygen | binding event | |
| | cleavable e-tags | | |
| | Sandwich assays | | Hydrogen Peroxide |
| | Antibody-1 decorated with | | (H₂O₂) |
| | Glucose oxidase while antibody- | | |
| | 2 | | |
| | is decorated with hydrogen | | |
| | peroxide cleavable e-tags | | |
| | Competition assays | | |
| | Antibody-1 decorated with | | |
| | Glucose oxidase while antibody- | | |
| | 2 is decorated with hydrogen | | |
| | peroxide cleavable e-tags | | |
| Patches in | Sandwich assays | | Light; Enzymes, |
| microfluidic | Antibody-1 is attached to a solid | | singlet oxygen, |
| channels; | surface while antibody-2 is | | hydrogen peroxide |
| integrated assay and | decorated with cleavable e-tags | | fluoride, reducing |
| separation device | Competition assays | | agents, mass |
| | Antibody-1 is attached to a solid | | spectra, others |
| | surface while antibody-2 is | | |
| | decorated with cleavable e-tags | | |

The assays may be performed in a competitive mode or a sandwich mode. In the competitive mode, the target competes with a labeled binding member for the reciprocal member. The reciprocal member is bound to the support, either during the complex formation or after, e.g. where an antibody is a specific binding member and anti-immunoglobulin is the reciprocal binding member and is bound to the support. In this mode, the binding sites of the reciprocal binding member become at least partially filled by the target, reducing the number of available binding sites for the labeled reciprocal binding member. Thus, the number of labeled binding members that bind to the reciprocal binding member will be in direct proportion to the number of target molecules present. In the sandwich mode, the target is able to bind at the same time to different binding members; a first support bound member and a second member that binds at a site of the target molecule different from the site at which the support bound member binds. The resulting complex has three components, where the target serves to link the labeled binding member to the support.

In carrying out the assays, the components are combined, usually with the target composition added first and then the labeled members in the competitive mode and in any order in the sandwich mode. Usually, the labeled member in the competitive mode will be equal to at least about 50% of the highest number of target molecules anticipated, preferably at least equal and may be in about 2 to about 10 fold excess or greater. The particular ratio of target molecules to labeled molecules will depend on the binding affinities, the length of time the mixture is incubated, the off rates for the target molecule with its reciprocal binding member, the size of the sample and the like. In the case of the sandwich assays, one will have at least an equal amount of the labeled binding member to the highest expected amount of the target molecules, usually at least about 1.5 fold excess, more usually at least about 2 fold excess and may have about 10 fold excess or more. The components are combined under binding conditions, usually in an aqueous medium, generally at a pH in the range of about 5-about 10, with buffer at a concentration in the range of about 10 to about 200 mM. These conditions are conventional, where conventional buffers may be used, such as phosphate, carbonate, HEPES, MOPS, Tris, borate, etc., as well as other conventional additives, such as salts, stabilizers, organic solvents, etc.

Usually, the unbound labeled binding member or e-tag probe will be removed by washing the bound labeled binding member. Where particles or beads are employed, these may be separated from the supernatant before washing, by filtration, centrifugation, magnetic separation, etc. After washing, the support may be combined with a liquid into which the e-tag reporters are to be released and/or the functionality of the e-tags is reacted with the detectable label, followed by or preceded by release. Depending on the nature of the cleavable bond and the method of cleavage, the liquid may include reagents for the cleavage. Where reagents for cleavage are not required, the liquid is conveniently an electrophoretic buffer. For example, where the cleavable linkage is photo labile, the support may be irradiated with light of appropriate wavelength to release the e-tag reporters. Where detectable labels are not present on the e-tags, the e-tags may be reacted with detectable labels. In some instances the detectable label may be part of the reagent cleaving the cleavable bond, e.g. a disulfide with a thiol. Where there is a plurality of different functionalities on different binding members for reaction with the label, the different labels will have functionalities that react with one of the functionalities. The different labels may be added together or individually in a sequential manner. For example, where the functionalities involve thiols, carboxyl groups, aldehydes and olefins, the labels could have activated olefins, alcohols, amines and thiol groups, respectively. By having removable protective groups for one or more of the functionalities, the protective groups may be removed stepwise and the labels added stepwise. In this way cross-reactivity may be avoided. Whether one has the detectable label present initially or one adds the detectable label is not critical to this invention and will frequently be governed by the nature of the target composition, the nature of the labeled binding members, and the nature of the detectable labels. For the most part, it will be a matter of convenience as to the particular method one chooses for providing the detectable label on the e-tag.

Where a reagent is necessary for cleavage, the e-tag reporters may be required to be separated from the reagent solution, where the reagent interferes with the electrophoretic analysis. Depending on the nature of the e-tag reporters and the reagent, one may sequester the e-tag reporters from the reagent by using ion exchange columns, liquid chromatography, an initial electrophoretic separation, and the like.

Alternatively, a capture ligand may be employed bound to the e-tag or retained portion of the target-binding moiety for isolating the e-tag probe, so as to remove any interfering components in the mixture. As used herein, the term “capture ligand,” refers to a group that is typically included within the target-binding moiety portion of an e-tag probe and is capable of binding specifically to a “capture agent” or receptor. The interaction between such a capture ligand and the corresponding capture agent may be used to separate uncleaved e-tag probes from released e-tag reporters. If desired, the receptor may be used to physically sequester the molecules to which it binds, entirely removing intact e-tag probes containing the target-binding region or modified target-binding regions retaining the ligand. These modified target-binding regions may be as a result of degradation of the starting material, contaminants during the preparation, aberrant cleavage, etc., or other nonspecific degradation products of the target binding sequence. As above, a ligand, exemplified by biotin, is attached to the target-binding region, e.g., the penultimate nucleoside, so as to be separated from the e-tag reporter upon cleavage. Other reagents that are useful include a ligand-modified nucleotide and its receptor. Ligands and receptors include biotin and streptavidin, ligand and antiligand, e.g. digoxin or derivative thereof and antidigoxin, etc. By having a ligand conjugated to the oligonucleotide, one can sequester the eTag conjugated oligonucleotide probe and its target with the receptor, remove unhybridized eTag reporter conjugated oligonucleotide and then release the bound eTag reporters or bind an oppositely charged receptor, so that the ligand-receptor complex with the eTag reporter migrates in the opposite direction.

In one exemplary use of capture ligands, a snp detection sequence may be further modified to improve separation and detection of the released e-tags. By virtue of the difference in mobility of the e-tags, the snp detection sequences will also have different mobilities. Furthermore, these molecules will be present in much larger amounts than the released e-tags, so that they may obscure detection of the released e-tags. Also, it is desirable to have negatively charged snp detection sequence molecules, since they provide for higher enzymatic activity and decrease capillary wall interaction. Therefore, by providing that the intact snp detection sequence molecule can be modified with a positively charged moiety, but not the released e-tag, one can change the electrostatic nature of the snp detection sequence molecules during the separation. By providing for a capture ligand on the snp detection sequence molecule to which a positively charged molecule can bind, one need only add the positively charged molecule to change the electrostatic nature of the snp detection sequence molecule. Conveniently, one will usually have a ligand of under about 1 kDa. This may be exemplified by the use of biotin as the ligand and avidin, which is highly positively charged, as the receptor (capture agent)/positively charged molecule. Instead of biotin/avidin, one may have other pairs, where the receptor, e.g. antibody, is naturally positively charged or is made so by conjugation with one or more positively charged entities, such as arginine, lysine or histidine, ammonium, etc. The presence of the positively charged moiety has many advantages in substantially removing the snp detection sequence molecules.

If desired, the receptor may be used to physically sequester the molecules to which it binds, removing entirely intact e-tags containing the target-binding moiety or modified target-binding moieties retaining the ligand. These modified target-binding moieties may be as a result of degradation of the starting material, contaminants during the preparation, aberrant cleavage, etc., or other nonspecific degradation products of the target binding sequence. As above, a ligand, exemplified by biotin, is attached to the target-binding moiety, e.g. the penultimate nucleoside, so as to be separated from the e-tag upon cleavage.

After a 5′ nuclease assay, a receptor for the ligand, for biotin exemplified by streptavidin (hereafter “avidin”) is added to the assay mixture (Example 10). Other receptors include natural or synthetic receptors, such as immunoglobulins, lectins, enzymes, etc. Desirably, the receptor is positively charged, naturally as in the case of avidin, or is made so, by the addition of a positively charged moiety or moieties, such as ammonium groups, basic amino acids, etc. Avidin binds to the biotin attached to the detection probe and its degradation products. Avidin is positively charged, while the cleaved electrophoretic tag is negatively charged. Thus the separation of the cleaved electrophoretic tag from, not only uncleaved probe, but also its degradation products, is easily achieved by using conventional separation methods. Alternatively, the receptor may be bound to a solid support or high molecular weight macromolecule, such as a vessel wall, particles including beads, e.g. magnetic particles, cellulose, agarose, etc., and separated by physical separation or centrifugation, dialysis, etc. This method further enhances the specificity of the assay and allows for a higher degree of multiplexing.

As a general matter, one may have two ligands, if the nature of the target-binding moiety permits. As described above, one ligand can be used for sequestering e-tags bound to the target-binding moiety, retaining the first ligand from products lacking the first ligand. Isolation and concentration of the e-tags bound to a modified target-binding moiety lacking the first ligand would then be performed. In using the two ligands, one would first combine the reaction mixture with a first receptor for the first ligand for removing target-binding moiety retaining the first ligand. One could either separate the first receptor from the composition or the first receptor would be retained in the composition, as described. This would be followed by combining the resulting composition, where the target-binding moiety containing the first ligand is bound to the first receptor, with the second receptor, which would serve to isolate or enrich for modified target-binding moiety lacking the first ligand, but retaining the second ligand. The second ligand could be the detectable label; a small molecule for which a receptor is available, e.g. a hapten, or a portion of the e-tag could serve as the second ligand. After the product is isolated or enriched, the e-tag could be released by denaturation of the receptor, displacement of the product, high salt concentrations and/or organic solvents, etc.

For e-tags associated with nucleic acid sequences, improvements include employing a blocking linkage between nucleotides in the sequence, particularly at least one of the links between the second to fourth nucleotides to inhibit cleavage at this or subsequent sites, and using control sequences for quantitation. Further improvements in the e-tags provide for having a positively multicharged moiety joined to the e-tag probe during separation.

While the ligand may be present at a position other than the penultimate position and one may make the ultimate linkage nuclease resistant, so that cleavage is directed to the penultimate linkage, this will not be as efficient as having cleavage at the ultimate linkage.

The above are generally applicable not only to generating a single e-tag per sequence detected, but also to generation of a single oligonucleotide fragment for fragment separation and identification by electrophoresis or by mass spectra, as it is essential to get one fragment per sequence detected. For purpose of explanation, these methods are illustrated below. FIGS. 3A-C provide a schematic illustration of the generalized methods of the invention employing a nucleotide target and a 5′ exonuclease indicating that only one eTag is generated per target for maximum multiplexing capabilities.

Once the solution of e-tag reporters is prepared and free of any interfering components, the solution may be analyzed electrophoretically. The analysis may employ capillary electrophoresis devices, microfluidic devices or other devices that can separate a plurality of compounds electrophoretically, providing resolved bands of the individual e-tag reporters.

The protocols for the subject homogeneous assays will follow the procedures for the analogous heterogeneous assays, which may or may not include a releasable e-tag. These protocols employ a signal producing system that includes the label on one of the binding members, the cleavable bond associated with the e-tag, electromagnetic radiation or other reagents involved in the reaction or for diminishing background signal. In assays involving the production of hydrogen peroxide, one may wish to have a molecule in solution that degrades hydrogen peroxide to prevent reaction between hydrogen peroxide produced by a label bound to an analyte molecule and an e-tag labeled binding member that is not bound to the same analyte molecule.

Generally, the concentrations of the various agents involved with the signal producing system will vary with the concentration range of the individual analytes in the samples to be analyzed, generally being in the range of about 10 nM to about 10 mM. Buffers will ordinarily be employed at a concentration in the range of about 10 to about 200 mM. The concentration of each analyte will generally be in the range of about 1 pM to about 100 μM, more usually in the range of about 100 pM to about 10 μM. In specific situations the concentrations may be higher or lower, depending on the nature of the analyte, the affinity of the reciprocal binding members, the efficiency of release of the e-tag reporters, the sensitivity with which the e-tags are detected, and the number of analytes, as well as other considerations.

The reactive species that is produced in the assay, analogous to the subject assay, is employed in a different way than was used in the analogous assay, but otherwise the conditions will be comparable. In many instances, the chemiluminescent compound when activated will result in cleavage of a bond, so that one may obtain release of the e-tag reporter. Assays that find use are described in U.S. Pat. Nos. 4,233,402, 5,616,719, 5,807,675, and 6,002,000. One would combine the analyte with one or both reagents. The particular order of addition will vary with the nature of the reagents. Generally, the binding reagents and the sample are combined and the mixture is allowed to incubate, generally at least about 5 min, more usually at least about 15 min, before irradiating the mixture or adding the remaining reagents.

The subject libraries of e-tags may be used to analyze the effect of an agent on a plurality of different compounds. For example, a plurality of substrates labeled with an e-tag may be prepared, where the enzyme catalyzes a reaction resulting in a change in mobility between the product and the starting material. These assays can find use in determining affinity groups or preferred substrates for hydrolases, oxidoreductases, lyases, etc. For example, with kinases and phosphatases, a charged group is added or removed so as to change the mobility of the product. By preparing a plurality of alcohols or phosphate esters, a determination may be made concerning which of the compounds serves as a substrate. By labeling the substrates with e-tags, the shift from the substrate to the product can be observed as evidence of the activity of a candidate substrate with the enzyme. By preparing compounds as suicide inhibitors, the enzymes may be sequestered and the e-tag reporters released to define those compounds that may serve as suicide inhibitors and, therefore, preferentially bind to the active site of the enzyme.

The subject methods may be used for screening for the activity of one or more candidate compounds, particularly drugs, for their activity against a battery of enzymes. In this situation, active substrates for each of the enzymes to be evaluated may be used, where each of the substrates has its own e-tag. For those enzymes for which the drug is an inhibitor, the amount of product is diminished in relation to the amount of product in the absence of the candidate compound. In each case the product has a different mobility from the substrate, so that the substrates and products can be readily distinguished by electrophoresis. By appropriate choice of substrates and detectable labels, electropherograms may be obtained showing the effect of the candidate compound on the activity of the different enzymes.

In determinations involving nucleic acids, since snp detection is, for the most part, the most stringent in its requirements, most of the description is directed toward the multiplexed detection of snp's by way of example and not limitation. For other nucleic acid analyses, frequently the protocols will be substantially the same, although in some instances somewhat different protocols are employed for snp's, because of the greater demands snp's make on fidelity. For proteins, the protocols are substantially different and are described independently of the snp protocols.

A. Primer Extension Reaction in Nucleic Acid Analyses

The extension reaction is performed by bringing together the necessary combination of reagents, and subjecting the mixture to conditions for carrying out the desired primer extension. Such conditions depend on the nature of the extension, e.g., PCR, single primer amplification, LCR, NASBA, 3SR and so forth, where the enzyme that is used for the extension has 5′-3′ nuclease activity. The extension reaction may be carried out as to both strands or as to only a single strand. Where pairs of primer and snp detection sequence are used for both strands, conveniently, the e-tag is the same but the bases are different. In this situation, a cleavable linkage to the base is employed, so that for the same snp, the same e-tag is obtained. Alternatively, if the number of snp's to be determined is not too high, different e-tags can be used for each of the strands. Usually, the reaction is carried out by using amplifying conditions, so as to provide an amplified signal for each snp. Amplification conditions normally employ thermal cycling, where after the primer extension and release of electrophoretic tag reporters associated with snp's which are present, the mixture is heated to denature the double-stranded DNA, cooled, where the primer and snp detection sequence can rehybridize and the extension be repeated.

Reagents for conducting the primer extension are substantially the same reaction materials for carrying out an amplification, such as an amplification indicated above. The nature and amounts of these reagents are dependent on the type of amplification conducted. In addition to oligonucleotide primers, the reagents also comprise nucleoside triphosphates and a nucleotide polymerase having 5′-3′ nuclease activity. The conditions for the various amplification procedures are well known to those skilled in the art. In a number of amplification procedures, thermal cycling conditions as discussed above are employed to amplify the polynucleotides. The combination of reagents is subjected to conditions under which the oligonucleotide primer hybridizes to the priming sequence of, and is extended along, the corresponding polynucleotide. The exact temperatures can be varied depending on the salt concentration, pH, solvents used length of and composition of the target polynucleotide sequence and the oligonucleotide primers. Thermal cycling conditions are employed for conducting an amplification involving temperature or thermal cycling and primer extension such as in PCR or single primer amplification, and the like.

B. The Invader™ Reaction in Nucleic Acid Analyses

In one SNP determination protocol, the primer includes the complementary base of the SNP. This protocol is referred to as Invader™ technology, and is described in U.S. Pat. No. 6,001,567. The protocol involves providing: (a) (i) a cleavage means, which is normally an enzyme, referred to as a cleavase, that recognizes a triplex consisting of the target sequence, a primer which binds to the target sequence and terminates at the SNP position and a labeled probe that binds immediately adjacent to the primer and is displaced from the target at the SNP position, when a SNP is present. The cleavase clips the labeled probe at the site of displacement, releasing the label, (ii) a source of target nucleic acid, the target nucleic acid having a first region, a second region and a third region, wherein the first region is downstream from the second region and the second region is contiguous to and downstream from the third region, and (iii) first and second oligonucleotides having 3′ and 5′ portions, wherein the 3′ portion of the first oligonucleotide contains a sequence complementary to the third region of the target nucleic acid and the 5′ portion of the first oligonucleotide and the 3′ portion of the second oligonucleotide each contain sequences usually fully complementary to the second region of the target nucleic acid, and the 5′ portion of the second oligonucleotide contains sequence complementary to the first region of said target nucleic acid; (b) mixing, in any order, the cleavage means, the target nucleic acid, and the first and second oligonucleotides under hybridization conditions that at least the 3′ portion of the first oligonucleotide is annealed to the target nucleic acid and at least the 5′ portion of the second oligonucleotide is annealed to any target nucleic acid to from a cleavage structure, where the combined melting temperature of the complementary regions within the 5′ and 3′ portions of the first oligonucleotide when annealed to the target nucleic acid is greater than the melting temperature of the 3′ portion of the first oligonucleotide and cleavage of the cleavage structure occurs to generate labeled products; and (c) detecting the labeled cleavage products.

Thus, in an Invader assay, attachment of an e-tag to the 5′ end of the detector sequence results in the formation of an e-tag-labeled nucleotide when the target sequence is present. The e-tag labeled nucleotide is separated and detected. By having a different e-tag for each nucleic acid sequence of interest, each having a different electrophoretic mobility, one can readily determine the snp's or measure multiple sequences, which are present in a sample. The e-tags may require further treatment, depending on the total number of snp's or target sequences being detected.

C. Fluorescent Quenching

If desired, the snp detection e-tag probe may have a combination of a quencher and a fluorescer. In this instance the fluorescer would be in proximity to the nucleoside to which the linker is bonded, as well as the quencher, so that in the primer extension mixture, fluorescence from fluorescer bound to the snp detection sequence would be quenched. As the reaction proceeds and fluorescer is released from the snp detection sequence and, therefore, removed from the quencher, it would then be capable of fluorescence. By monitoring the primer extension mixture for fluorescence, a determination may be made as to determine when there would probably be a sufficient amount of individual e-tags to provide a detectable signal for analysis. In this way, time and reagent may be saved by terminating the primer extension reaction at the appropriate time. There are many quenchers that are not fluorescers, so as to minimize fluorescent background from the snp detection sequence. Alternatively, one could take small aliquots and monitor the reaction for detectable e-tag reporters.

D. Analysis of Reaction Products

The separation of the e-tag reporters by electrophoresis can be performed in conventional ways. See, for example, U.S. Pat. Nos. 5,750,015, 5,866,345, 5,935,401, 6,103,199, and 6,110,343, and WO98/5269, and references cited therein. Also, the sample can be prepared for mass spectrometry in conventional ways. See, for example, U.S. Pat. Nos. 5,965,363, 6,043,031, 6,057,543, and 6,111,251. As mentioned above, in one embodiment of the invention the fluorescer is the same for all members of a set of e-tag reporters. However, it is within the scope of the invention to employ sets of fluorescers where each set comprises the same fluorescer with different mobility modifiers for the members of the set but the fluorescers are different among the sets. Depending on current instrumentation, from one to four different fluorescers activated by the same light source and emitting at different detectable labels may be used. With improvements, five or more different fluorescers will be available, where an additional light source may be required. Electrochemical detection is described in U.S. Pat. No. 6,045,676. In one embodiment involving primer extension, after completion of the primer extension reaction, either by monitoring the change in fluorescence as described above or taking aliquots and assaying for total free e-tags, the mixture may be analyzed.

The presence of each of the cleaved e-tags is determined by the label. The separation of the mixture of labeled e-tag reporters is typically carried out by electroseparation, which involves the separation of components in a liquid by application of an electric field, preferably, by electrokinesis (electrokinetic flow) electrophoretic flow, or electroosmotic flow, or combinations thereof, with the separation of the e-tag reporter mixture into individual fractions or bands. Electroseparation involves the migration and separation of molecules in an electric field based on differences in mobility. Various forms of electroseparation include, by way of example and not limitation, free zone electrophoresis, gel electrophoresis, isoelectric focusing and isotachophoresis. Capillary electroseparation involves electroseparation, preferably by electrokinetic flow, including electrophoretic, dielectrophoretic and/or electroosmotic flow, conducted in a tube or channel of about 1-200 micrometer, usually, about 10-100 micrometers cross-sectional dimensions. The capillary may be a long independent capillary tube or a channel in a wafer or film comprised of silicon, quartz, glass or plastic.

In capillary electroseparation, an aliquot of the reaction mixture containing the e-tag products is subjected to electroseparation by introducing the aliquot into an electroseparation channel. In the case of nucleic acid determination, the channel may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan.

For a homogeneous assay, the sample, e-tag-labeled probe mixture, and ancillary reagents are combined in a reaction mixture supporting the cleavage of the linking region. The mixture may be processed to separate the e-tag reporters from the other components of the mixture. The mixture, with or without e-tag reporter enrichment, may then be transferred to an electrophoresis device, usually a microfluidic or capillary electrophoresis device and the medium modified as required for the electrophoretic separation. Where it is desired to remove from the separation channel intact e-tag reporter molecules, a ligand is bound to the e-tag that is not released when the e-tag reporter is released. Alternatively, by adding a reciprocal binding member that has the opposite charge of the e-tag reporter, so that the overall charge is opposite to the charge of the e-tag reporter, these molecules will migrate toward the opposite electrode from the released e-tag reporter molecules.

Capillary devices are known for carrying out amplification reactions such as PCR. See, for example, Analytical Chemistry (1996) 68:4081-4086. Devices are also known that provide functional integration of PCR amplification and capillary electrophoresis in a microfabricated DNA analysis device. One such device is described by Woolley, et al., in Anal. Chem. (1996) 68:4081-4086. The device provides a microfabricated silicon PCR reactor and glass capillary electrophoresis chips. In the device a PCR chamber and a capillary electrophoresis chip are directly linked through a photolithographically fabricated channel filled with a sieving matrix such as hydroxyethylcellulose. Electrophoretic injection directly from the PCR chamber through the cross injection channel is used as an “electrophoretic valve” to couple the PCR and capillary electrophoresis devices on a chip.

The capillary electrophoresis chip contains a sufficient number of main or secondary electrophoretic channels to receive the desired number of aliquots from the PCR reaction medium or the solutions containing the cleaved labels, etc., at the intervals chosen.

For capillary electrophoresis one or more detection zones may be employed to detect the separated cleaved labels. It is, of course, within the purview of the present invention to utilize several detection zones depending on the nature of the amplification process, the number of cycles for which a measurement is to be made and so forth. There may be any number of detection zones associated with a single channel or with multiple channels. Suitable detectors for use in the detection zones include, by way of example, photomultiplier tubes, photodiodes, photodiode arrays, avalanche photodiodes, linear and array charge coupled device (CCD) chips, CCD camera modules, spectrofluorometers, and the like. Excitation sources include, for example, filtered lamps, LED's, laser diodes, gas, liquid and solid-state lasers, and so forth. The detection may be laser scanned excitation, CCD camera detection, coaxial fiber optics, confocal back or forward fluorescence detection in single or array configurations, and the like.

Detection may be by any of the known methods associated with the analysis of capillary electrophoresis columns including the methods shown in U.S. Pat. No. 5,560,811 (column 11, lines 19-30), U.S. Pat. Nos. 4,675,300, 4,274,240 and 5,324,401, the relevant disclosures of which are incorporated herein by reference.

Those skilled in the electrophoresis arts will recognize a wide range of electric potentials or field strengths may be used, for example, fields of about 10 to about 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

IV. Kits for Use of the e-tags

As a matter of convenience, predetermined amounts of reagents employed in the present invention can be provided in a kit in packaged combination. The kit comprises a set of electrophoretic tag (e-tag) probes for detecting the binding of or interaction between each or any of a plurality of ligands and one or more target antiligands. The set comprises j members, and each of the e-tag probes has the form Mⱼ—D—L—Tⱼ, wherein Mⱼ, D, L, and Tⱼ are as defined above. The kit may further comprise a device for conducting capillary electrophoresis. The kit can further include various buffered media, some of which may contain one or more of the above reagents.

One exemplary kit for snp detection can comprise in packaged combination an oligonucleotide primer for each polynucleotide suspected of being in said set wherein each of said primers is hybridizable to a first sequence of a respective polynucleotide if present, a template dependent polynucleotide polymerase, nucleoside triphosphates, and a set of primer and oligonucleotide snp detection sequences, each of the snp detection sequences having a fluorescent label at its 5′-end and having a sequence at its 5′-end that is hybridizable to a respective polynucleotide wherein each of the electrophoretic labels is cleavable from the snp detection sequence.

The relative amounts of the various reagents in the kits can be varied widely to provide for concentrations of the reagents necessary to achieve the objects of the present invention. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. Each reagent can be packaged in separate containers or some reagents can be combined in one container where cross-reactivity and shelf life permit. For example, the dNTPs, the oligonucleotide pairs, optionally the polymerase, may be included in a single container, which may also include an appropriate amount of buffer. The kits may also include a written description of a method in accordance with the present invention as described above.

### EXAMPLES

The invention is demonstrated further by the following illustrative examples. Parts and percentages are by weight unless otherwise indicated. Temperatures are in degrees Centigrade (°C.) unless otherwise specified. The following preparations and examples illustrate the invention but are not intended to limit its scope. Unless otherwise indicated, oligonucleotides and peptides used in the following examples were prepared by synthesis using an automated synthesizer and were purified by gel electrophoresis or HPLC.

The following abbreviations have the meanings set forth below:

Tris HCl—Tris(hydroxymethyl)aminomethane-HCl (a 10×solution) from Bio Whittaker, Walkersville, Md.

HPLC—high performance liquid chromatography

BSA—bovine serum albumin from Sigma Chemical Company, St. Louis Mo.

EDTA—ethylene diamine tetra-acetate from Sigma Chemical Company

bp—base pairs

g—grams

mM—millimolar

TET—tetrachlorofluorescein

FAM—fluorescein

TAMRA—tetramethyl rhodamine

EOF—electroosmotic flow

Reagents

All reagents were synthesized as described below or purchased from Aldrich Chemical Company, Milwaukee Wis., with the exception of 6-carboxyfluorescein, which was purchased from Molecular Probes, Eugene Oreg., and 2-cyanoethyl diisopropylchlorophosphoramidite, which was purchased from Chem Genes, Ashland Mass.

### Example 1

Synthesis of AMD 001

Into a 250 mL round bottom flask was placed 2-methyl resorcinol (6.46 g, 52.0 mmol) and trimellitic anhydride (5.0 g, 26.0 mmol). Methanesulfonic acid (50 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine precipitate was filtered and dried to afford AMD001 (2.0 g, 90%). Mass (LR ES⁻) calculated for C₂₃H₁₆O₇ 404, found: 403 (M−H+).

### Example 2

Synthesis of AMD 002

Into a 50 mL round bottom flask was put 4-chlororesorcinol (1.5 g, 10.4 mmol), trimellitic anhydride (1.0 g, 5.2 mmol), and zinc chloride (0.36 g, 2.6 mmol). This mixture of solids was heated to 175° C. using an oil bath. At elevated temperatures all materials liquefied and subsequently turned dark in color. After allowing the reaction to stir for 30 min, the solution was cooled to room temperature and then dissolved in a 15% NaOH solution. The dark red solution was acidified with a 50% HCl solution which resulted in the formation of an orange solid which was filtered and dried to yield AMD002 (1.4 g, 60%). Mass (LR ES⁻) calculated for C₂₁H₁₀Cl₂O₇ 544, found: 543 (M−H+).

### Example 3

Synthesis of AMD 003

Into a 50 mL round bottom flask was put 4-ethyl resorcinol (1.44 g, 10.4 mmol) and trimellitic anhydride (1.0 g, 5.2 mmol). Methanesulfonic acid (10 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine orange precipitate was filtered and dried to afford AMD003 (2.0 g, 87%). Mass (LR ES⁻) calculated for C₂₅H₂₀O₇ 432, found: 431 (M−H+). Mass (LR ES⁻) calculated for C₂₁H₁₀Cl₂O₇ 544, found: 543 (M−H+).

### Example 4

Synthesis of AMD 004

Into a 100 mL round bottom flask was put 4-hexylresorcinol (2.0 g, 10.4 mmol) and trimellitic anhydride (1.0 g, 5.2 mmol). Methanesulfonic acid (15 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine orange precipitate was filtered and dried to afford AMD004 (2.2 g, 78%). Mass (LR ES⁻) calculated for C₃₃H₃₆O₇ 544, found: 543 (M−H+).

### Example 5

Synthesis of AMD 005

Into a 50 mL round bottom flask was put 2,4-dihydroxybenzoic acid (1.6 g, 10.4 mmol) and trimellitic anhydride (1.0 g, 5.2 mmol). Methanesulfonic acid (10 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine orange precipitate was filtered and dried to afford AMD005 (2.1 g, 88%). Mass (LR ES⁻) calculated for C₂₃H₁₂O₁₁ , 464, found: 463 (M−H+).

### Example 6

Synthesis of AMD 006

Into a 50 mL round bottom flask was put 2,6-dihydroxybenzoic acid (1.6 g, 10.4 mmol) and trimellitic anhydride (1.0 g, 5.2 mmol). Methanesulfonic acid (10 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine orange precipitate was filtered and dried to afford AMD006 (2.3 g, 96%). Mass (LR ES⁻) calculated for C₂₃H₁₂O₁₁ , 464, found: 463 (M−H+).

### Example 7

Synthesis of AMD 007

Into a 50 mL round bottom flask was put 2,5-dimethylresorcinol (1.0 g, 7.24 mmol) and trimellitic anhydride (0.7 g, 3.6 mmol). Methanesulfonic acid (10 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine orange precipitate was filtered and dried to afford AMD007 (0.65 g, 42%). Mass (LR ES⁻) calculated for C₂₅H₂₀O₇ 432, found: 431 (M−H+).

### Example 8

Synthesis of AMD 008

Synthesis of 2,4-dihydroxy-2′,4′ or 5′-dicarboxybenzophenone—Into a 50 mL round bottom flask was put 5(6)-carboxyfluorescein (10 g, 26.6 mmol) and 10 mL of water containing 18 g of sodium hydroxide. The suspension was heated to 175° C. in an oil bath for 2 hours then diluted with 50 ml of water and allowed to cool to room temperature. Acidification with concentrated hydrochloric acid precipitated the product as a tan solid (7 g, 93%).

Into a 25 mL pear bottom flask was put 1,3-dihydroxynaphthalene (0.5 g, 3.12 mmol) and 2,4-dihydroxy-2′,4′ or 5′-dicarboxybenzophenone (0.9 g, 3.12 mmol) (prepared as described above). Methanesulfonic acid (5 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (50 mL). The resulting fine dark precipitate was filtered and dried to afford AMD 008 (0.2 g, 13%). Mass (LR ES⁻) calculated for C₂₅H₁₄O₇ 426, found: 425 (M−H+).

### Example 9

Synthesis of AMD 009

Into a 25 mL round bottom flask was put 4-chlororesorcinol (0.18 g, 1.27 mmol) (prepared as described above) and 2,4-dihydroxy-2′,4′ or 5′-dicarboxybenzophenone (0.36 g, 1.27 mmol). (1.0 g, 5.2 mmol). Methanesulfonic acid (5 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (50 mL). The resulting fine dark precipitate was filtered and dried to afford AMD 009 (0.05 g, 10%). Mass (LR ES⁻) calculated for C₂₁H₁₁ClO₇ 410, found: 409 (M−H+).

### Example 10

Synthesis of AMD 012

Into a 10 mL round bottom flask was put 1,3-dihydroxynaphthalene (0.17 g, 1.04 mmol) and trimellitic anhydride (0.1 g, 3.5 mmol). Methanesulfonic acid (2 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (10 mL). The resulting dark precipitate was filtered and dried to afford AMD 012 (0.05 g, 20%). Mass (LR ES⁻) calculated for C₂₉H₁₆O₇ 476, found: 475 (M−H+).

### Example 11

Synthesis of AMD-S 001

Synthesis of 2-methyl-4-chlororesorcinol—The reaction scheme for this synthesis is depicted in FIG. 5. Into a 500 mL round bottom flask was put 2-methylresorcinol (10.0 g, 80.6 mmol) and diethyl ether (150 mL). This solution was stirred under an atmosphere of nitrogen and cooled to 0° C. with an ice/methanol bath. Sulfuryl chloride was dissolved in 50 mL of diethyl ether and added dropwise from an addition funnel over a period of one hour. After complete addition, the solution was allowed to warm to room temperature and stirring was continued for three hours. The reaction was neutralized with a saturated solution of sodium bicarbonate, the organic phase washed with water (2×100 mL) then brine (2×100 mL) and dried over Na₂SO₄, filtered, and concentrated in vacuo to yield a yellow oil. On standing, this set up to light brown crystals of 2-methyl-4-chlororesorcinol (10 g, 78%).

Synthesis of dichlorodimethylfluorescein (AMD-S 001)—The reaction scheme for this synthesis is depicted in FIGS. 5-6. Into a 50 mL round bottom flask was put 2-methyl-4-chlororesorcinol (1.66 g, 10.4 mmol), trimellitic anhydride (1.0 g, 5.2 mmol), and zinc chloride (0.36 g, 2.6 mmol). This mixture of solids was heated to 175° C. using an oil bath. At elevated temperatures all materials liquefied and subsequently turned dark in color. After allowing the reaction to stir for 30 min, the solution was cooled to room temperature and then dissolved in a 15% NaOH solution. The dark red solution was acidified with a 50% HCl solution, which resulted in the formation of an orange solid which was filtered and dried to yield AMD-S 001 (1.3 g, 52%). Mass (LR ES⁻) calculated for C₂₃H₁₄Cl₂O₇ 472, found: 471 (M−H+).

### Example 12

Synthesis of AMD-S 002

Synthesis of dichlorotrimellitic acid—The reaction scheme for this synthesis is depicted in FIG. 7. Into a 250 mL round bottom flask was put 2,5-dichloroxylene (5 g, 28.6 mmol) and aluminum chloride (4.6 g, 34.3 mmol). Solids were thoroughly mixed, then acetyl chloride (2.0 mL, 28.6 mmol) was added and the reaction immersed in an oil bath at 70° C. After cessation of gas evolution (approx 30 min), reaction was allowed to cool to room temperature and partitioned between ethyl acetate (100 mL) and water (200 mL). The organic layer was dried over Na₂SO₄, filtered, and concentrated in vacuo to yield 5 g of 6-acetyl-2,5-dichloroxylene as a yellow oil. This crude was used directly for the subsequent oxidation without purification.

Into a 250 mL round bottom flask was put 6-acetyl-2,5-dichloroxylene (5 g) and 100 mL of a 10% solution of potassium carbonate containing 18 g of potassium permanganate. This was heated to 110° C. in an oil bath for 4 hours, after which time it was cooled to room temperature and poured into 6N H2SO4. Manganese dioxide settled out of solution, but was not filtered. Aqueous phase was extracted with diethyl ether (3×100 mL) and the pooled extracts washed once with brine (200 ml), dried over Na₂SO₄, filtered, and concentrated in vacuo to yield dichlorotrimellitic acid (2.92 g, 37% overall) as a white solid. Mass (LR ES⁻) calculated for C₉H₄Cl₂O₆ 278, found: 277 (M−H+), 555 (2M−H+).

Synthesis of tetrachlorodimethylfluorescein (AMD-S 002)—The reaction scheme for this synthesis is depicted in FIG. 8. Into a 50 mL round bottom flask was put 2-methyl4-chlororesorcinol (2.84 g, 17.9 mmol) (prepared as described above), and 2,5-dichlorotrimellitic acid (2.5 g, 9.0 mmol) (prepared as described above). Methanesulfonic acid (15 mL) was then added and the resulting suspension was heated to 130° C. using an oil bath. At elevated temperatures all materials went into solution, which subsequently turned dark in color. After allowing the reaction to stir for 30 min, the solution was cooled to room temperature and then added dropwise to rapidly stirring water (100 mL). The resulting fine red precipitate was filtered and dried to afford AMD-S 002 (4.5 g, 90%). Mass (LR ES⁻) calculated for C₂₃H₁₂Cl₄O₇ 540, found: 539 (M−H+).

### Example 13

Isolation of Fluorescein Derivatives

A. General Procedure for the Isolation of 6-carboxyfluorescein Derivatives

Fluorescein derivatives, prepared as a mixture of 5(6) isomers, were added to 100 equivalents of acetic anhydride and 4 equivalents of pyridine. This solution was heated briefly and monitored by TLC 45:45:10 (Hxn:EtoAc:MeOH) for the formation of the diacetyl derivative. After complete conversion, the solution was stored at 4° C. overnight to precipitate the 6-carboxy pyridinium salt, which was filtered and washed with additional acetic anhydride. This diacetyl salt was converted back to the free fluorescein derivative by treatment with ammonia in methanol.

B. Isolation of 6-carboxydichlorodimethylfluorescein

Into a 50 mL round bottom flask was put 5(6)-carboxy dichlorodimethylfluorescein (1 g, 2.1 mmol), acetic anhydride (19.8 mL, 210 mmol), and pyridine (0.681 mL, 8.4 mmol). Heated to 100° C. in an oil bath for 10 minutes then stored at 4° C. overnight. Precipitate which had formed was filtered and washed with 5 mL cold acetic anhydride. This off white salt (465 mg) was suspended in 1 mL of methanol and added to 2 mL of a 7N solution of ammonia in methanol. The salt immediately dissolved and the solution became dark orange in color. Evaporation of solvent and recrystallization of the residue from methanol gave 200 mg of 6-carboxy dichlorodimethylfluorescein a crimson red solid.

### Example 14

Synthesis of Elements of e-tag Probes

A. Synthesis of 6-Carboxyfluorescein Phosphoramidite Derivatives

To a solution of 6-carboxyfluorescein (6-FAM) (0.5 g, 1.32 mmol) in dry pyridine (5 mL) was added drop wise, isobutyric anhydride (0.55 mL, 3.3 mmol). The reaction was allowed to stir at room temperature under an atmosphere of nitrogen for 3 h. After removal of pyridine in vacuo the residue was redissolved in ethyl acetate (150 mL) and washed with water (150 mL). The organic layer was separated, dried over Na₂SO₄, filtered, and concentrated in vacuo to yield a brownish residue. This material was dissolved in CH₂Cl₂ (5 mL) after which N-hydroxy succinimide (0.23 g, 2.0 mmol) and dicyclohexylcarbodiimide (0.41 g, 1.32 mmol) were added. The reaction was allowed to stir at room temperature for 3 h and then filtered through a fritted funnel to remove the white solid, which had formed. To the filtrate was added aminoethanol (0.12 mL, 2.0 mmol) dissolved in 1 mL of CH₂Cl₂. After 3 h the reaction was again filtered to remove a solid that had formed, and then diluted with additional CH₂Cl₂ (50 mL). The solution was washed with water (150 mL) and then separated. The organic layer was dried over Na₂SO₄, filtered, and concentrated in vacuo to yield a white foam (0.7 g, 95%, 3 steps). ¹H NMR: (DMSO), 8.68 (t, 1H), 8.21 (d, 1H), 8.14 (d, 1H), 7.83 (s, 1H), 7.31 (s, 2H), 6.95 (s, 4H), 4.69 (t, 1H), 3.45 (q, 2H), 3.25 (q, 2H), 2.84 (h, 2H), 1.25 (d, 12 H). Mass (LR FAB⁺) calculated for C₃₁H₂₉NO₉ (M+H⁺) 559.2, found: 560.

B. Synthesis of Modified Fluorescein Phosphoramidites

Pivaloyl protected carboxyfluorescein: Into a 50 mL round bottom flask was placed 5(6)-carboxyfluorescein (0.94 g, 2.5 mmol), potassium carbonate (1.0 g, 7.5 mmol) and 20 mL of dry DMF. The reaction was stirred under nitrogen for 10 min, after which trimethylacetic anhydride (1.1 mL, 5.5 mmol) was added via syringe. The reaction was stirred at room temperature overnight, and then filtered to remove excess potassium carbonate and finally poured into 50 mL of 10% HCl. A sticky yellow solid precipitated out of solution. The aqueous solution was decanted off and the residual solid was dissolved in 10 mL of methanol. Drop wise addition of this solution to 10% HCl yielded a fine yellow precipitate, which was filtered and air dried to yield an off white solid (0.88 g, 62%). TLC (45:45:10 of Hxn:EtOAc:MeOH).

NHS ester of protected pivaloyl carboxyfluorescein. Into a 200 mL round bottom flask was placed the protected carboxyfluorescein (2.77 g, 5.1 mmol) and 50 mL of dichloromethane. N-hydroxysuccinimide (0.88 g, 7.6 mmol) and dicyclohexylcarbodiimide (1.57 g, 7.6 mmol) were added and the reaction was stirred at room temperature for 3 hours. The reaction was then filtered to remove the precipitated dicyclohexyl urea byproduct and reduced to approx. 10 mL of solvent in vacuo. Drop wise addition of hexanes with cooling produced a yellow-orange colored solid, which was triturated with hexanes, filtered and air-dried to yield 3.17 g (95%) of product. TLC (45:45:10 of Hxn:EtOAc:MeOH)

Alcohol. Into a 100 mL round bottom flask was placed the NHS ester (0.86 g, 1.34 mmol) and 25 mL of dichloromethane. The solution was stirred under nitrogen after which aminoethanol (81 mL, 1 eq) was added via syringe. The reaction was monitored by TLC (45:45:10 Hxn, EtOAc, MeOH) and was found to be complete after 10 min. The dichloromethane was then removed in vacuo and the residue dissolved in EtOAc, filtered and absorbed onto 1 g of silica gel. This was bedded onto a 50 g silica column and eluted with Hxn:EtOAc:MeOH (9:9:1) to give 125 mg (20%) of clean product.

Phosphoramidite. Into a 10 mL round bottom flask containing 125 mg of the alcohol was added 5 mL of dichloromethane. Diisopropyl ethylamine (139 μL, 0.8 mmol) was added via syringe. The colorless solution turned bright yellow. 2-cyanoethyl diisopropylchlorophosphoramidite (81 μL, 0.34 mmol) was added via syringe and the solution immediately went colorless. After 1 hour TLC (45:45:10 of Hxn:EtOAc:TEA) showed the reaction was complete with the formation of two closely eluting isomers. Material was purified on a silica column (45:45:10 of Hxn:EtOAc:TEA) isolating both isomers together and yielding 130 mg (85%).

Carboxylic acid. Into a 4 mL vial was placed 12-aminododecanoic acid (0.1 g, 0.5 mmol) and 2 mL of pyridine. To this suspension was added chlorotrimethyl silane (69 μL, 1.1 eq) via syringe. After all material dissolved (10 min) NHS ester (210 mg, 0.66 eq) was added. The reaction was stirred at room temperature overnight and then poured into water to precipitate a yellow solid, which was filtered, washed with water, and air-dried. TLC (45:45:10 of Hxn:EtOAc:MeOH) shows a mixture of two isomers.

General Procedure for Remaining Syntheses. The carboxylic acid formed as described above is activated by NHS ester formation with 1.5 eq each of N-hydroxysuccinimide and dicyclohexylcarbodiimide in dichloromethane. After filtration of the resulting dicyclohexylurea, treatment with 1 eq of varying amino alcohols will effect amide bond formation and result in a terminal alcohol. Phosphitylation using standard conditions described above will provide the phosphoramidite.

C. Synthesis on an ABI 394 DNA Synthesizer

6-Carboxyfluorescein (6-FAM) phosphoramidite is prepared by the addition of 2.96 ml of anhydrous acetonitrile to a 0.25 gram bottle of the fluorescein phosphoramidite, to give a 0.1 M solution. The bottle is then loaded onto the ABI 394 DNA synthesizer at position 8 using the standard bottle change protocol. The other natural [dA^{bz} (0.1 M: 0.25 g/2.91 mL anhydrous acetonitrile), dC^{Ac}(0.1 M: 0.25 g/3.24 mL anhydrous acetonitrile), dT(0.1 M: 0.25 g/3.36 mL anhydrous acetonitrile), dG^{dmf} (0.1 M: 0.25 g/2.81 mL anhydrous acetonitrile)] phosphoramidite monomers are loaded in a similar fashion to ports 1-4. Acetonitrile is loaded onto side port 18, standard tetrazole activator is loaded onto port 9, CAP A is loaded onto port 11, CAP B is loaded onto port 12, oxidant is loaded onto port 15, and deblock solution is loaded onto port 14 all using standard bottle change protocols.

Standard Reagents Employed for DNA Synthesis

Oxidizer: 0.02 M Iodine (0.015 M for MGB Probes)

DeBlock: 3% trichloracetic acid in dichloromethane

Activator: 1H-Tetrazole in anhydrous acetonitrile

HPLC Grade Acetonitrile (0.002% water)

Cap A: Acetic Anhydride

Cap B: N-methyl imidazole

The target sequence of interest is then input with a terminal coupling from port 8 to attach ACLA 001 to the 5′-end of the sequence. A modified cycle is then chosen such that the desired scale (0.2 μmol, 1.0 μmol, etc.) of DNA is synthesized. The modified cycle contains an additional wait step of 800 seconds after any addition of 6-FAM. A standard DNA synthesis column containing the support upon which the DNA will be assembled is then loaded onto one of four positions of the DNA synthesizer. DNA containing e-tag reporters have been synthesized on various standard 500 Å CPG supports (Pac-dA-CPG, dmf-dG-CPG, Ac-dC-CPG, dT-CPG) as well as specialty supports containing 3′-biotin, 3′-amino linker, and minor grove binding species.

Upon completion of the synthesis, the column is removed from the synthesizer and either dried under vacuum or by blowing air or nitrogen through the column to remove residual acetonitrile. The column is then opened and the CPG is removed and placed in a 1-dram vial. Concentrated ammonia is added (2.0 mL) and the vial is sealed and placed into a heat block set at 65° C. for a minimum of two hours. After two hours the vial is allowed to cool to room temperature after which the ammonia solution is removed using a Pasteur pipette and placed into a 1.5 mL Eppendorf tube. The solution is concentrated in vacuo and submitted for HPLC purification.

D. Synthesis of Phosphoramidites of AMD-S001 and AMD-S002

The phosphoramidites of AMD-S001 and AMD-S002 were synthesized in a manner similar to that described above for the synthesis of the phosphoramidite of 6-FAM.

### Example 15

Electroseparation of Fluorescent Compound Conjugates on Microfluidic Chip

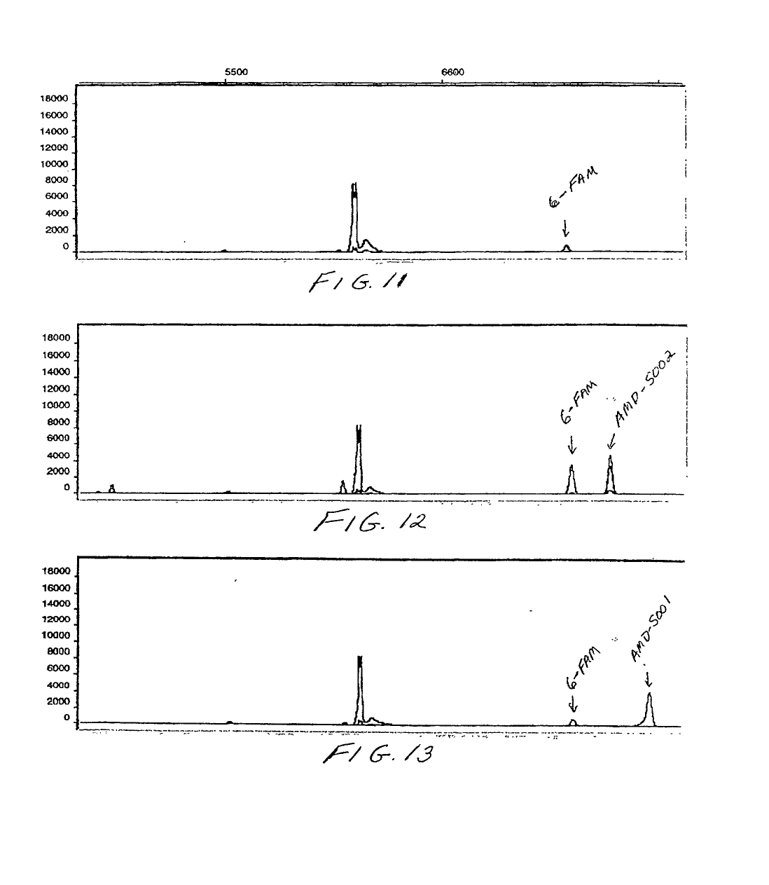

Phosphoramidites comprising AMD-S001 and AMD-S002 and 6-FAM prepared as described above were combined in an aqueous buffered solution and were separated and detected in an electrophoresis chip. Detection was 0.5 cm for the injection point on the anodal side of an electrophoresis channel. The results are shown in FIG. 10 (6-FAM alone), FIG. 11 (6-FAM and AMD-S001) and FIG. 12 (6-FAM and AMD-S002).

It is evident from the above results that the subject inventions provide powerful ways of preparing compositions for use in multiplexed determinations and methods for performing multiplexed determinations using such compositions. The methods provide for homogeneous and heterogeneous protocols, both with nucleic acids and proteins, as exemplary of other classes of compounds. It is further evident from the above results that the subject invention provides an accurate, efficient and sensitive process, as well as compositions for use in the process, to perform multiplexed reactions. The protocols provide for great flexibility in the manner in which determinations are carried out and maybe applied to a wide variety of situations involving haptens, antigens, nucleic acids, cells, etc., where one may simultaneously perform a number of determinations on a single or plurality of samples and interrogate the samples for a plurality of events. The results of the determination are readily read in a simple manner using electrophoresis or mass spectrometry. Systems are provided where the entire process, after addition of the sample and reagents, may be performed under the control of a data processor with the results automatically recorded.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

It is claimed:

1. In a method for separating and detecting molecules comprising separating the molecules on the basis of different mobilities and detecting the separated molecules by the presence therein of a luminescent moiety, the improvement comprising the luminescent moieties of all of the molecules having substantially the same spectral properties and differing from one another by the presence therein of a modification that imparts a different mobility to each of the respective molecules.

2. The method of claim 1 wherein the luminescent moieties are excited at the same wavelength and emission therefrom is detected at the same wavelength.

3. The method of claim 1 wherein the luminescent moieties are fluorescent moieties.

4. The method of claim 1 wherein the luminescent moieties are all derived by modifications of the same parent luminescent moiety.

5. The method of claim 1 wherein the modification is selected from the group comprising alkylene groups, alkylenoxy groups, amino acids, and nucleotide groups.

6. The method of claim 1 wherein the spectral properties are emission, absorption and quantum yield.

7. The method of claim 1 wherein the molecules are selected from the group consisting of polypeptides and polynucleotides.

8. A set of electrophoretic tag (e-tag) probes for detecting the binding of or interaction between each or any of a plurality of ligands and one or more target antiligands, the set comprising j members, and each of said e-tag probes having the form:
Mⱼ—D—L—Tⱼ,
wherein
(a) D is a detection group comprising a detectable label that is the same for all of said e-tag probes;
(b) Tⱼ is a ligand capable of binding to or interacting with a target antiligand,
(c) L is a bond or a linking group linking D and Tⱼ and comprising a cleavable linkage at the point of attachment to D or within L at a point that is common to all of said e-tag probes, wherein cleavage of said cleavable linkage produces an e-tag reporter of the form Mⱼ—D or Mⱼ—D—L′, where L′ is the residue of L attached to Mⱼ—D after such cleavage, and
(d) Mⱼ is a mobility modifier having a charge/mass ratio that imparts a unique and known electrophoretic mobility to a corresponding e-tag reporter, within a selected range of electrophoretic mobilities with respect to other e-tag reporters of the same form in the probe set.

9. The probe set of claim 8 wherein the luminescent molecule is a fluorescent molecule.

10. The probe set of claim 8 wherein the luminescent molecule is selected from the group consisting of fluorescein, substituted fluorescein, rhodamine and substituted rhodamine.

11. The probe set of claim 8 wherein the modification is selected from the group comprising alkylene groups, alkylenoxy groups, amino acids, and nucleotide groups.

12. The probe set of claim 8 wherein the spectral properties are emission, absorption and quantum yield.

13. The probe set of claim 8 wherein Tⱼ are selected from the group consisting of polypeptides and polynucleotides.

14. The probe set of claim 8, wherein L includes at least a portion of an amino acid sequence that is recognized and cleaved by a selected peptidase.

15 The probe set of claim 8, wherein L includes at least a portion of an oligosaccharide that is recognized and cleaved by a selected hydrolytic enzyme.

16. The probe set of claim 8, wherein L comprises an ester linkage that is cleaved by a selected esterase.

17. The probe set of claim 8, wherein L comprises a disulfide bond, and the antiligand is attached to an oxidase enzyme, such that in the presence of a substrate for the enzyme, H₂O₂ generated by the oxidase is effective to cleave the disulfide linkage in a probe bound to the antiligand.

18. The probe of claim 8, wherein L comprises a bond cleavable by singlet oxygen, wherein the antiligand is attached to a sensitizer capable of generating singlet oxygen.

19. The probe set of claim 8, for use in screening for a ligand capable of binding to a receptor, wherein the ligands are represented by Tⱼ and are members of a combinatorial library of small organic molecules, and the antiligand is the receptor.

20. The probe set of claim 8, for use in screening for a ligand capable of binding to a receptor, wherein the ligands are represented by Tⱼ and are members of a combinatorial library of nucleotide sequences, and the antiligand is the receptor and is a polynucleotide.

21. The probe set of claim 8, wherein each Mⱼ has a unique charge/mass ratio by virtue of variations in mass, but not charge.

22. The probe set of claim 8, wherein each Mⱼ has a unique charge/mass ratio, by virtue of changes in both mass and charge.

23. The probe set claim 8, wherein each Mⱼ is formed of a selected number of negatively charged and/or positively charged amino acids.

24. The probe set of claim 8, wherein each Mⱼ includes an alkyl chain, and differs from other Mⱼ in the set by 1-3 methylene groups in the chain.

25. The probe set of claim 8, wherein each Mⱼ includes an alkylene oxide chain, and differs from other Mⱼ in the set by 1-3 methylene groups in the chain.

26. The probe set of claim 8, wherein each Mⱼ includes a combination of an alkylene oxide chain and an alkylene chain, and differs from other Mⱼ in the set by 1-3 methylene groups in the chain.

27. A method for detecting the binding of or interaction between a first binding agent and each and any of a plurality of second binding agents, comprising:
(a) subjecting a mixture comprising the first binding agent and the second binding agents to conditions for interaction there between, wherein the second binding agent comprises a cleavable reporter group, where the cleavable reporter group in each second binding agent includes:
(i) a cleavable moiety, and
(ii) at least one tag, wherein the at least one tag has a detectable moiety and a mobility unique to the second binding agent,
wherein all of the at least one tags in the second binding agents have the same spectral properties and
(b) subjecting said mixture to conditions for releasing said cleavable reporter group;
(c) separating the released reporter groups by their differences in mobility; and
(d) detecting the binding of or interaction between a first binding agent and each second binding agent based on the unique mobility of the corresponding reporter group, wherein all of the at least one tags are excited at a single wavelength and emission therefrom is detected at a single wavelength.

28. The method of claim 27 wherein the at least one tag in each of the second binding agents is derived from the same luminescent molecule and wherein the tags differ among the second binding agents by virtue of a modification that imparts a unique mobility without altering the spectral properties of the luminescent molecule.

29. The method of claim 27 wherein the luminescent molecule is a fluorescent molecule.

30. The method of claim 27 wherein the luminescent molecule is selected from the group consisting of fluorescein, substituted fluorescein, rhodamine and substituted rhodamine.

31. The method of claim 27 wherein the modification is selected from the group comprising alkylene groups, alkylenoxy groups, amino acids and nucleotide groups.

32. The method of claim 27 wherein the spectral properties are emission, absorption and quantum yield.

33. The method of claim 27 wherein a set of electrophoretic tag (e-tag) probes is employed, the set comprising j members, and each of said e-tag probes having the form:
Mⱼ—D—L—Tⱼ,
wherein
(a) D is a detection group comprising a detectable label that is the same for all of said e-tag probes;
(b) Tⱼ is a ligand capable of binding to or interacting with a target antiligand,
(c) L is a bond or a linking group linking D and Tⱼ and comprising a cleavable linkage at the point of attachment to D or within L at a point that is common to all of said e-tag probes, wherein cleavage of said cleavable linkage produces an e-tag reporter of the form Mⱼ—D or Mⱼ—D—L′, where L′ is the residue of L attached to Mⱼ—D after such cleavage, and
(d) Mⱼ is a mobility modifier.

34. The method of claim 33 wherein the luminescent molecule is a fluorescent molecule.

35. The method of claim 34 wherein the luminescent molecule is selected from the group consisting of fluorescein, substituted fluorescein, rhodamine and substituted rhodamine.

36. The method of claim 34 wherein the modification is selected from the group comprising alkylene groups, alkylenoxy groups, amino acids and nucleotide groups.

37. The method of claim 33 wherein the spectral properties are emission, absorption and quantum yield.

38. The method of claim 33 wherein Tⱼ are selected from the group consisting of polypeptides and polynucleotides.

39. The method of claim 27, wherein the mobility modifier has a charge/mass ratio that imparts a unique and known electrophoretic mobility to a corresponding e-tag reporter, within a selected range of electrophoretic mobilities with respect to other e-tag reporters of the same form in the probe set and wherein the reporters are separated according to the electrophoretic mobility imparted by their charge/mass ratio.

40. The method of claim 27, wherein the mobility modifier having a mass that imparts a unique and known mass to a corresponding reporter, within a selected range of masses with respect to other reporters of the same form in the probe set and wherein the reporters are separated by mass spectrometry.

41. The method of claim 27, for use in detecting the binding/interaction of each of a plurality of ligands with a ligand receptor, wherein the receptor forms the first binding agent and the ligands form the second binding agents.

42. The method of claim 41, for use in detecting the binding/interaction of each of a plurality of ligands with a ligand receptor, wherein
(a) the receptor forms the first binding agent,
(b) the ligands form the second binding agents, and
(c) a plurality of third binding agents are combined with the first and second binding agents in the generating step, wherein each second binding agent has a corresponding third binding agent that is capable of binding uniquely to the corresponding second binding agent in a manner that does not interfere with binding between the first and second binding agents, and wherein each third binding agent has covalently bound thereto, a ligand-specific cleavable reporter group.

43. The method of claim 42, wherein the third binding agents are antibodies.

44. The method of claim 27, wherein Tⱼ is a target-binding moiety that is a polynucleotide or a polypeptide.

45. A set of electrophoretic tag (e-tag) probes for detecting the binding of or interaction between each or any of a plurality of ligands and one or more target antiligands, the set comprising j members, and each of said e-tag probes having the form:
(D, Mⱼ)—L—Tⱼ,
where
(a) D is a detection group comprising a detectable label;
(b) Tⱼ is a ligand capable of binding to or interacting with a target antiligand,
(c) L is a linking group connected to Tⱼ by a bond that is cleavable by a selected cleaving agent when the probe is bound to or interacting with the target antiligand, wherein cleavage by said agent produces an e-tag reporter of the form (D, Mⱼ)—L′, where L′ is the residue of L attached to (D, Mⱼ) after such cleavage,
(d) Mⱼ is a mobility modifier that imparts a unique and known electrophoretic mobility to a corresponding e-tag reporter of the form (D, Mⱼ)—L′, within a selected range of electrophoretic mobilities with respect to other e-tag reporters of the same form in the probe set; and
(e) (D, Mⱼ)— includes both D—Mⱼ— and Mⱼ—D—;
wherein at least two detectable labels are employed and are independently selected from compounds having substantially the same spectral properties and of the formula: 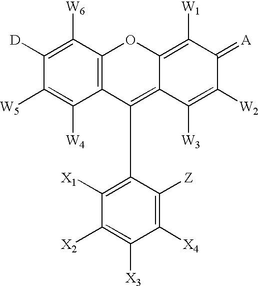
wherein:
Z is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, phosphorus containing species, or polymer chains of from about 2 to about 10 monomer units,
A is O, N⁺(R¹)(R²) wherein R¹ and R² are independently H, lower alkyl, or substituted lower alkyl,
D is OH, OR³ wherein R³ is lower alkyl, substituted lower alkyl, aryl, substituted aryl, N(R¹)(R²) wherein R¹ and R² are independently H, lower alkyl, or substituted lower alkyl,
W¹, W², W³, W⁴, W⁵ and W⁶ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
X¹-X⁴ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
wherein W² and W³ may be taken together to form one or more rings comprising 4 to 14 atoms and comprising 1 to 7 unsaturations, and
wherein W⁴ and W⁵ may be taken together to form a ring comprising 4 to 14 atoms and comprising 1 to 7 unsaturations.

46. The probe set of claim 45 wherein said detectable labels are independently selected from compounds having substantially the same spectral properties and of the formula: 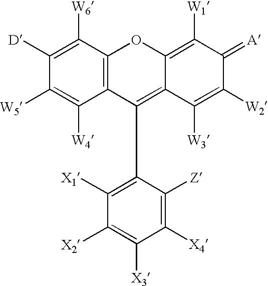
wherein:
Z′ is COOH,
A′ is O,
D′ is OH, OR^{3′} wherein R^{3′} is lower alkyl, substituted lower alkyl, aryl, or substituted aryl,
W^{1′}, W^{2′}, W^{3′}, W^{4′} and W^{6′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
W^{5′} is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, chlorine, fluorine, bromine, iodine, COOH, carboxylate, amide, nitrile, nitro, sulfonyl, sulfate, sulfone, amino, tethered amino, quaternary amino, or imino,
X^{1′}-X^{4′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
wherein W^{2′} and W^{3′} may be taken together to form one or more rings comprising 4 to 14 atoms and comprising 1 to 7 unsaturations, and
wherein W^{4′} and W^{5′} may be taken together to form a ring comprising 4 to 14 atoms and comprising 1 to 7 unsaturations.

47. The probe set of claim 45 wherein said detectable labels are independently selected from compounds having substantially the same spectral properties and of the formula: 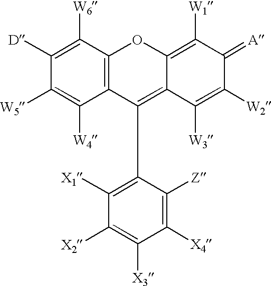
wherein
Z″ is COOH,
A″ is O, N(R^{1″})(R^{2″}) wherein R^{1″} and R^{2″} are independently lower alkyl, or substituted lower alkyl,
D″ is OH, OR^{3″} wherein R^{3″} is lower alkyl, substituted lower alkyl, aryl, or substituted aryl,
W^{1″} and W^{6″} are independently H, lower alkyl, substituted lower alkyl, COOH, chloro, or fluoro,
W^{2″} and W^{5″} are independently H, lower alkyl, substituted lower alkyl, COOH, chloro, or fluoro,
W^{3″} and W^{4″} are independently H, lower alkyl, substituted lower alkyl, COOH, chloro, or fluoro,
wherein W^{2″} and W^{3″} may be taken together to form one or more rings comprising 4 to 14 atoms and comprising 1 to 7 unsaturations, and
wherein W^{4″} and W^{5″} may be taken together to form a ring comprising 4 to 14 atoms and comprising 1 to 7 unsaturations,
X^{1″}-X^{4″} are independently H, chloro, fluoro, COOH, bromo, or iodo.

48. The probe set of claim 47 wherein said detectable labels are independently selected from compounds having substantially the same spectral properties and of the formula wherein Z″ is carboxyl, W^{6″} and W^{1″} are lower alkyl, W^{5″} and W^{2″} are halogen, X^{2″} and X^{3″} are hydrogen or carboxyl and X^{1″} and X^{4″} are hydrogen or halogen.

49. The probe set of claim 47 wherein said detectable labels are independently selected from compounds having substantially the same spectral properties and of the formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are hydrogen.

50. The probe set of claim 47 wherein said detectable labels are independently selected from compounds having substantially the same spectral properties and of the formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are chloro.

51. The probe set of claim 45 wherein said detectable labels are independently a compound of FIG. 1 having the same spectral properties.

52. The probe set of claim 45, wherein Tⱼ is a target-binding moiety that is a polynucleotide or a polypeptide.

53. A method for detecting the binding of or interaction between a first binding agent and each and any of a plurality of second binding agents, comprising:
(a) subjecting a mixture comprising the first binding agent and the second binding agents to conditions for interaction therebetween, wherein the second binding agent comprises a cleavable reporter group, where the cleavable reporter group in each second binding agent includes:
(i) a cleavable moiety, and
(ii) at least one tag, wherein the at least one tag has a detectable moiety and a mobility unique to the second binding agent,
wherein at least two detectable labels are employed and are independently selected from compounds having substantially the same spectral properties and of the formula: 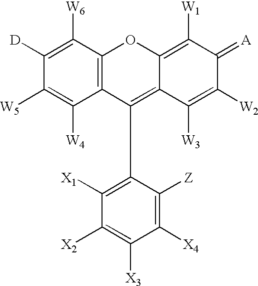
wherein:
Z is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, imino, phosphorus containing species, or polymer chains of from about 2 to about 10 monomer units,
A is O, N⁺(R¹)(R²) wherein R¹ and R² are independently H, lower alkyl, or substituted lower alkyl,
D is OH, OR³ wherein R³ is lower alkyl, substituted lower alkyl, aryl, substituted aryl, N(R¹)(R²) wherein R¹ and R² are independently H, lower alkyl, or substituted lower alkyl,
W¹, W², W³, W⁴, W⁵ and W⁶ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
X¹-X⁴ are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
wherein W² and W³ may be taken together to form one or more rings comprising 4 to 14 atoms and comprising 1 to 7 unsaturations, and
wherein W⁴ and W⁵ may be taken together to form a ring comprising 4 to 14 atoms and comprising 1 to 7 unsaturations.
(b) subjecting said mixture to conditions for releasing said cleavable reporter group;
(c) separating the released reporter groups by their differences in mobility; and
(d) detecting the binding of or interaction between a first binding agent and each second binding agent based on the unique mobility of the corresponding reporter group.

54. The method of claim 53 wherein said detectable labels are independently selected from compounds having substantially the same spectral properties and of the formula: 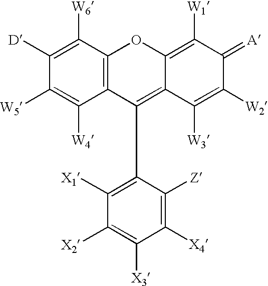
wherein:
Z′ is COOH,
A′ is O,
D′ is OH, OR^{3′} wherein R^{3′} is lower alkyl, substituted lower alkyl, aryl, or substituted aryl,
W^{1′}, W^{2′}, W^{3′}, W^{4′} and W^{6′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
W^{5′} is H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, chlorine, fluorine, bromine, iodine, COOH, carboxylate, amide, nitrile, nitro, sulfonyl, sulfate, sulfone, amino, tethered amino, quaternary amino, or imino,
X^{1′}-X^{4′} are independently H, lower alkyl, substituted lower alkyl, lower alkenyl, substituted lower alkenyl, lower alkynyl, substituted lower alkynyl, cycloalkyl, alkoxy, substituted alkoxy, phenoxy, substituted phenoxy, aromatic, substituted aromatic, phenyl, substituted phenyl, polycyclic aromatic, substituted polycyclic aromatic, heterocyclic, substituted heterocyclic, chlorine, fluorine, bromine, iodine, COOH, carboxylate, amide, nitrile, nitro, sulfonyl, sulfate, sulfone, amino, tethered amino, quaternary amino, or imino,
wherein W^{2′} and W^{3′} may be taken together to form one or more rings comprising 4 to 14 atoms and comprising 1 to 7 unsaturations, and
wherein W^{4′} and W^{5′} may be taken together to form a ring comprising 4 to 14 atoms and comprising 1 to 7 unsaturations.

55. The method of claim 53 wherein said detectable labels are independently selected from compounds having substantially the same spectral properties and of the formula: 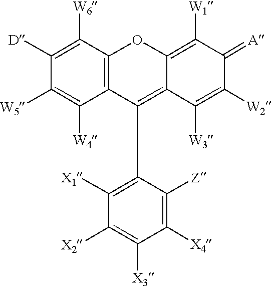
wherein
Z″ is COOH,
A″ is O, N(R^{1″})(R^{2″}) wherein R^{1″} and R^{2″} are independently lower alkyl, or substituted lower alkyl,
D″ is OH, OR^{3″} wherein R^{3″} is lower alkyl, substituted lower alkyl, aryl, or substituted aryl,
W^{1″} and W^{6″} are independently H, lower alkyl, substituted lower alkyl, COOH, chloro, or fluoro,
W^{2″} and W^{5″} are independently H, lower alkyl, substituted lower alkyl, COOH, chloro, or fluoro,
W^{3″} and W^{4″} are independently H, lower alkyl, substituted lower alkyl, COOH, chloro, or fluoro,
wherein W^{2″} and W^{3″} may be taken together to form one or more rings comprising 4 to 14 atoms and comprising 1 to 7 unsaturations, and
wherein W^{4″} and W^{5″} may be taken together to form a ring comprising 4 to 14 atoms and comprising 1 to 7 unsaturations,
X^{1″}-X^{4″} are independently H, chloro, fluoro, COOH, bromo, or iodo.

56. The method of claim 55 wherein said detectable labels are independently selected from compounds having substantially the same spectral properties and of the formula wherein Z″ is carboxyl, W^{6″} and W^{1″} are lower alkyl, W^{5″} and W^{2″} are halogen, X^{2″} and X^{3″} are hydrogen or carboxyl and X^{1″} and X^{4″} are hydrogen or halogen.

57. The method of claim 55 wherein said detectable labels are independently selected from compounds having substantially the same spectral properties and of the formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are hydrogen.

58. The method of claim 55 wherein said detectable labels are independently selected from compounds having substantially the same spectral properties and of the formula wherein Z″ is carboxyl, W^{6″} and W^{1″} are methyl, W^{5″} and W^{2″} are chloro, one of X^{2″} and X^{3″} are hydrogen and the other is carboxyl and X^{1″} and X^{4″} are chloro.

59. The method of claim 53 wherein said detectable labels are independently a compound of FIG. 1 having the same spectral properties.

60. The method of claim 53 wherein Tⱼ are selected from the group consisting of polypeptides and polynucleotides.

61. The method of claim 53, for use in detecting the binding/interaction of each of a plurality of ligands with a ligand receptor, wherein
(a) the receptor forms the first binding agent,
(b) the ligands form the second binding agents, and
(c) a plurality of third binding agents are combined with the first and second binding agents in the generating step, wherein each second binding agent has a corresponding third binding agent that is capable of binding uniquely to the corresponding second binding agent in a manner that does not interfere with binding between the first and second binding agents, and wherein each third binding agent has covalently bound thereto, a ligand-specific cleavable reporter group.

62. The method of claim 61, wherein the third binding agents are antibodies.

63. A kit for use in detecting the presence and/or amount of each and any of a plurality of bivalent target molecules, comprising in packaged combination:
(a) first binding agent (i) capable of binding to a first binding site on said target molecules, and
(b) a plurality of second binding agents, each capable of target-specific binding to a second binding site on a selected target, and each having a unique cleavable reporter group in each second binding agent that includes (i) a cleavable moiety that is susceptible to cleavage, and (ii) an electrophoretic tag selected from the set of electrophoretic tags of claim 45.

64. The kit of claim 63 wherein the first binding agent and the second binding agents are polynucleotides for detecting each and any of a plurality of target DNA sequences.

65. The kit of claim 63 wherein the first binding agent and the second binding agents are polypeptides for detecting each and any of a plurality of target polypeptides.

66. The kit of claim 63 wherein Tⱼ are selected from the group consisting of polypeptides and polynucleotides.

67. A set of electrophoretic tags, each member of said set comprising a mobility modifier, a detectable label and a target binding moiety wherein at least two fluorescent compounds are independently employed as detectable labels in said set wherein said fluorescent compounds have substantially the same spectral properties and different mass and charge.

68. A compound of the formula:
M′″-dN(Fl)-L^{b}—N
wherein Fl is a fluorescent compound, and dN is deoxynucleotide, N is a nucleotide, M′″ is an alkylene oxide chain and L^{b} is an alkylene oxide chain.

69. The compound of claim 68 wherein dN is dT, dC, dU, dG or dA.

70. The compound of claim 68 wherein N is T, C, U, G, or A.

71. The compound of claim 68, which is a compound set forth in FIGS. 14 and 15.
